(19) United States
(12) Patent Application Publication
(10) Pub. No.: US 2025/0051245 A1
(43) Pub. Date: Feb. 13, 2025

(54) METHOD FOR THE PREPARATION OF A COMPOSITION COMPRISING DISSOLVED [18F]FLUORIDE AND COMPOSITION OBTAINABLE BY THE METHOD

(71) Applicant: Technische Universität München, München (DE)

(72) Inventors: Daniel Di Carlo, Haimhausen (DE); Hans-Jürgen Wester, Schweitenkirchen (DE)

(21) Appl. No.: 18/709,254

(22) PCT Filed: Oct. 31, 2022
(86) PCT No.: PCT/EP22/80357

§ 371 Date: May 10, 2024

(30) Foreign Application Priority Data
Nov. 16, 2021 (EP) 21208421.4

(51) Int. Cl. C07B 59/00

(52) U.S. Cl. CPC C07B 59/004; C07B 2200/05

(57) ABSTRACT

Provided is a method for the preparation of a composition comprising dissolved [¹⁸F]fluoride ions which is suitable for radiofluorination, said method comprising the steps of: providing an aqueous solution comprising water and [¹⁸F]fluoride ions; passing the aqueous solution through a solid phase extraction device comprising an anion exchange resin in order to trap [¹⁸F]fluoride ions on the anion exchange resin and to separate the [¹⁸F]fluoride ions trapped on the anion exchange resin from water; eluting [¹⁸F]fluoride ions from the anion exchange resin by passing an elution composition comprising an organic solvent and a salt of an alkanoic acid through the solid phase extraction device; obtaining a composition as an eluate which comprises the organic solvent, the salt of the alkanoic acid, and dissolved [¹⁸F]fluoride ions. Moreover, provided are the composition comprising dissolved [¹⁸F]fluoride ions, and a method for the preparation of a radiofluorinated organic compound, which involves the preparation of the composition comprising dissolved [¹⁸F]fluoride ions.

The present invention relates to a method for the preparation of a composition comprising dissolved [¹⁸F]fluoride which can be used for an efficient radiofluorination of organic compounds, such as compounds which comprise a Silicon-based Fluoride Acceptor (SiFA) group, and to the composition which is obtainable by the method in accordance with the invention. Moreover, a method for the preparation of a radiofluorinated organic compound is provided.

### ¹⁸F in Nuclear Medicine

In the last few decades, positron emission tomography (PET) has emerged to an established diagnostic procedure with a constantly increasing relevance in nuclear medicine. In contrast to other common PET radionuclides like ¹¹C, ¹³N, ¹⁵O, ⁶⁴Cu and ⁶⁸Ga, ¹⁸F has always attracted a major interest principally due to its advantageous physical properties.¹ Among them, the convenient half-life (109.7 min) stands out for being long enough to allow for the radiosynthesis of even complex tracers and their distribution to smaller centers without an own production facility.¹⁻² Furthermore, the decay of ¹⁸F occurs predominantly by positron emission (97%) with a relatively low energy (649 keV), rendering this isotope an ideal candidate for high resolution PET imaging.^{1,3} Despite these outstanding characteristics, the rather challenging radiochemistry of ¹⁸F has always represented the crucial limitation to its widespread use, whereas ⁶⁸Ga-labeled radiopharmaceuticals paved their way with a simple kit-like radiolabeling strategy. However, a variety of exciting new approaches has initiated the shift to a broader use of radiofluorinated tracers in nuclear medicine.

### Silicon-Based Fluoride Acceptors as Novel ¹⁸F-Labeling Approach

Introduction of ¹⁸F in radiotracers usually occurs via nucleophilic substitution reactions in electron-poor aromatic and aliphatic systems with a suitable leaving group. Due to the low reactivity of [¹⁸F]fluoride, harsh reaction conditions are usually needed to generate the [¹⁸F]fluorine-carbon bond.⁴ This causes the formation of undesired by-products and consequently implies laborious tracer purification.⁴ The harsh labeling conditions also hinder the direct radiofluorination of complex biomolecules, which are often only accessible through the use of prosthetic groups.⁵ For these reasons, intense research efforts were dedicated to the development of novel ¹⁸F-labeling strategies. In 2006, Schirrmacher et al. reported an alternative ¹⁸F-labeling approach based on the isotopic exchange of natural ¹⁹F by radioactive ¹⁸F on the silicon atom of a so-called Silicon-based Fluoride Acceptor, as illustrated for example by the following scheme.⁶⁻⁷

$$\text{?}$$

$$\text{?} \text{indicates text missing or illegible when filed}$$

A key advantage of this method relies in the fact that neither special activation reagents nor elevated reaction temperatures are required during labeling, eliminating the need for subsequent high-performance liquid chromatography (HPLC) purification which would reduce the radiochemical yield (RCY) and the molar activity (Aₘ) of the resulting ¹⁸F-labeled tracer.⁸ In recent years, this approach has evolved into a rapid and efficient method for preparation of ¹⁸F-labeled PET ligands with high RCYs and Aₘs.^{5, 9}

### Radiofluorination of Silicon-Based Fluoride Acceptors

First attempts to radiolabel Silicon-based Fluoride Acceptor-bearing compounds were conducted using azeotropically dried [¹⁸F]fluoride.⁸ It was soon recognized that partial neutralization of the required base during activity preparation represented a prerequisite for efficient radiofluorination.⁸ Unfortunately, the exact amount of acid needed for the neutralization reaction was difficult to determine due to variable base adsorption on the drying vessel wall.⁸ As a consequence, RCYs for the radiofluorination of Silicon-based Fluoride Acceptor-bearing compounds were only reproducible to a limited extend.⁸ It was later on realized, that [¹⁸F]fluoride preparation according to the so-called Munich Method constituted the preferable technique in this context.⁸ Originally developed by Wessmann et al., the Munich Method consists of trapping aqueous [¹⁸F]fluoride on an anion-exchange resin, followed by on-cartridge drying of the activity using an anhydrous solvent and recovery of dried [¹⁸F]fluoride by means of an elution cocktail composed of [K⁺⊂2.2.2]OH⁻ in MeCN.^{10, P1} Applying this technique for the radiofluorination of Silicon-based Fluoride Acceptors allowed to reach two goals at once. On the one hand, preparation of dried [¹⁸F]fluoride by solid-phase extraction evaded the laborious and time-consuming azeotropic distillation procedure.¹⁰ On the other hand, partial neutralization of the eluate was easier to achieve due to the absence of adsorption effects.⁸ Wängler et al. were the first to exactly quantify the influence of eluate neutralization on the subsequent radiofluorination of a Silicon-based Fluoride Acceptor.⁸ The group adjusted the hydroxide-containing [¹⁸F]fluoride eluate by addition of oxalic acid and determined the highest radiochemical conversion (RCC) for a Silicon-based Fluoride Acceptor-bearing somatostatin ligand using [K⁺⊂2.2.2]OH⁻ and the acid in a molar ratio of 4.⁸ A similar observation was made by Wurzer et al. investigating the isotopic exchange reaction on the Silicon-based Fluoride Acceptor-bearing PSMA ligand ^{nat}Ga-rhPSMA-7 with the same labeling strategy.¹¹ Substantial ¹⁸F-incorporation was only reported when the molar ratio between [K⁺⊂2.2.2]OH⁻ and oxalic acid corresponded to 3.3-6.7.¹¹ Their elaborated radiofluorination protocol includes [¹⁸F]fluoride preparation by the Munich Method, neutralization of the eluate with oxalic acid, optimized reaction conditions for the isotopic exchange and final radiotracer work-up by means of solid-phase extraction as illustrated in FIG. 1.

In detail, the optimized radiofluorination procedure for Silicon-based Fluoride Acceptors established by Wurzer et al. consists of loading aqueous [¹⁸F]fluoride onto the Sep-Pak® QMA Carbonate (⁴⁶ mg sorbent weight, ²³⁰ μeqg⁻¹ ion exchange capacity) and subsequently drying the activity by rinsing the cartridge with air, MeCN (10 mL) and air.¹⁶ Recovery of dried [¹⁸F]fluoride is realized by inversely purging the cartridge with an elution cocktail containing a solution of KOH (83 μmol) and Kryptofix® 222 (91 μmol) in MeCN (500 μL).¹⁶ The eluate is afterwards partly neutralized by addition of oxalic acid (1 M in MeCN, 30 μL, 30 μmol) and subsequently diluted with the Silicon-based Fluoride Acceptor-bearing compound (1 mm in DMSO, 10-150 μL, 10-150 nmol).¹⁶ Labeling occurs for 5 min at rt followed by dilution of the reaction mixture with an acidic buffer (PBS, pH=3, 9 mL).^{12, 16} Purification is subsequently conducted via simple solid-phase extraction as unincorporated [¹⁸F]fluoride represents the only impurity that needs to be separated. The radiofluorinated compound is therefore retained onto an Oasis® HLB Plus Light cartridge (30 mg sorbent weight) and flushed with PBS (10 mL) and air.¹² A mixture of ethanol and water (1:1, v/v, 300 μL) allows finally the elution of the purified tracer.¹²

Although the use of Munich dried [¹⁸F]fluoride has emerged as method of choice for radiofluorination of Silicon-based Fluoride Acceptors, certain drawbacks remain. Most notably, the exact addition of oxalic acid for partial neutralization of the eluate remains a weak point affecting radiolabeling efficiency.^{8, 11} Furthermore, since the adjusted eluate still exhibits an alkaline character, radiofluorination of base-labile precursors appears to be out of reach. Another aspect concerns the application of Munich dried [¹⁸F]fluoride in clinical routine. Due to its toxicity, Kryptofix® 222 concentration has to be determined in final radiotracer formulations before administration.¹³ In addition, oxalic acid is not listed in the US- and European Pharmacopeia, so that additional toxicological assessments and quality control procedure of the final product are required before a corresponding production procedure will be accepted for GMP production of a radiopharmaceutical in the context of clinical trials.

Before this background, it was the aim of the present invention to provide a method for the efficient preparation of a composition comprising dissolved [¹⁸F]fluoride, which can be advantageously used for radiofluorination, and a composition which is obtainable by the method.

In particular, relevant objectives of the invention can be summarized as follows:
to provide a method which helps to avoid the need for a subsequent evaporation step after [¹⁸F]fluoride elution from the anion-exchange resin;
to reduce the overall duration for the preparation of the composition comprising the [¹⁸F]fluoride, resulting in increased RCYs for the subsequent radiofluorination reaction;
to provide a composition comprising [¹⁸F]fluoride in a form that is readily applicable for the radiofluorination of Silicon-based Fluoride Acceptor-bearing compounds without the need of further additives;
to provide a composition allowing a particularly efficient radiofluorination of Silicon-based Fluoride Acceptor-bearing compounds through heating of the reaction mixture;
to provide a composition allowing the radiofluorination of base-sensitive compounds bearing a Silicon-based Fluoride Acceptor.

To that extent, the invention provides, in accordance with a first aspect, a method for the preparation of a composition comprising dissolved [¹⁸F]fluoride ions, said method comprising the steps of
providing an aqueous solution comprising water and [¹⁸F]fluoride ions;
passing the aqueous solution through a solid phase extraction device comprising an anion exchange resin in order to trap [¹⁸F]fluoride ions on the anion exchange resin and to separate the [¹⁸F]fluoride ions trapped on the anion exchange resin from water;
eluting [¹⁸F]fluoride ions from the anion exchange resin by passing an elution composition comprising an organic solvent and a salt of an alkanoic acid through the solid phase extraction device;
obtaining, as an eluate, a composition which comprises the organic solvent, the salt of the alkanoic acid, and dissolved [¹⁸F]fluoride ions.

A second aspect of the invention relates to a method for the preparation of a radiofluorinated organic compound, wherein the method comprises the steps of
preparing a composition comprising an organic solvent, a salt of an alkanoic acid, and dissolved [¹⁸F]fluoride ions by the method in accordance with the first aspect of the invention; and
contacting an organic compound to be radiofluorinated with the composition thus prepared to allow the organic compound to undergo a radiofluorination reaction with a [¹⁸F]fluoride ion comprised in the composition.

As a variant of the method for the preparation of a radiofluorinated organic compound in accordance with the second aspect, the organic compound to be radiofluorinated can also be added to the elution composition comprising an organic solvent and a salt of an alkanoic acid used in the method in accordance with the first aspect of the invention. In accordance with this variant, the [¹⁸F]fluoride ions can be eluted from the anion exchange resin in the presence of the organic compound to be radiofluorinated.

In accordance with another aspect, the invention provides a composition which comprises an organic solvent, a salt of an alkanoic acid, and dissolved [¹⁸F]fluoride ions. It will be understood that such a composition can be advantageously obtained as a product by the method in accordance with the first aspect of the invention.

It has been found by the present inventors that the use of the elution composition defined herein allows the [¹⁸F]fluoride to be efficiently eluted from the anion exchange resin, without the need to rely on water as a (co) solvent. Additional steps for the removal of water or any other solvent which may hinder a subsequent radiofluorination reaction, such as an evaporation step, can be dispensed with. Moreover, the presence of a cation in the form of a cryptate is not required.

Furthermore, the considerably lower basicity of the anion of the alkanoic acid in comparison to the hydroxide applied in the Munich Method discussed above constitutes a significant advantage. In particular, the partial neutralization of the eluate prior to any radiofluorination reaction by addition of a defined amount of acid is no longer required. The eluate of the present invention is immediately applicable for radiofluorination, in particular for the radiofluorination of Silicon-based Fluoride Acceptor-bearing compounds, and can be even extended to ¹⁸F-labeling of base-sensitive compounds. A further advantage of the eluate composition comprising the dissolved [¹⁸F]fluoride which is provided in accordance with the invention resides in the fact that the eluate can be heated up in order to increase the reaction rate and thus the RCY of the subsequent radiofluorination reaction without affecting the structural integrity of the compound that is reacted with the fluoride contained in the eluate.

The following items provide a summary of the aspects of the invention and of preferred embodiments thereof.
1. A method for the preparation of a composition comprising dissolved [¹⁸F]fluoride ions, said method comprising the steps of
   providing an aqueous solution comprising water and [¹⁸F]fluoride ions;
   passing the aqueous solution through a solid phase extraction device comprising an anion exchange resin in order to trap [¹⁸F]fluoride ions on the anion exchange resin and to separate the [¹⁸F]fluoride ions trapped on the anion exchange resin from water;
   eluting [¹⁸F]fluoride ions from the anion exchange resin by passing an elution composition comprising an organic solvent and a salt of an alkanoic acid through the solid phase extraction device;
   obtaining a composition as an eluate which comprises the organic solvent, the salt of the alkanoic acid, and dissolved [¹⁸F]fluoride ions.
2. The method according to item 1, wherein the solid phase extraction device is a solid phase extraction column or a solid phase extraction cartridge.
3. The method according to item 1 or 2, wherein the anion exchange resin is a resin containing quaternary ammonium groups.
4. The method according to any one of items 1 to 3, which further comprises a step of purging the solid phase extraction device comprising the trapped [¹⁸F]fluoride ions on the anion exchange resin with a gas after the aqueous solution has been passed through the device.
5. The method according to item 4, wherein the gas is selected from air, nitrogen, helium, and argon, or from mixtures of two or more of these.
6. The method according to any one of items 1 to 5, which further comprises a step of rinsing the anion exchange resin comprising the trapped [¹⁸F]fluoride ions with an organic solvent prior to eluting [¹⁸F]fluoride ions from the anion exchange resin.
7. The method according to item 6, wherein the organic solvent used for rinsing the anion exchange resin is an anhydrous solvent.
8. The method according to item 6 or 7, wherein the organic solvent used for rinsing the anion exchange resin comprises or consists of a polar aprotic solvent, preferably a solvent selected from dimethyl sulfoxide (DMSO) and acetonitrile (MeCN), and more preferably dimethyl sulfoxide (DMSO).
9. The method according to any one of items 6 to 8, which further comprises, prior to the step of eluting [¹⁸F]fluoride ions, a step of purging the solid phase extraction device comprising the trapped [¹⁸F]fluoride ions on the anion exchange resin with a gas after the anion exchange resin has been rinsed with the organic solvent.

10. The method according to item 9, wherein the gas is selected from air, nitrogen, helium, and argon, or from mixtures of two or more of these.

11. The method according to any one of items 1 to 10, wherein the elution composition comprises a salt of an alkanoic acid represented by formula (A-1):

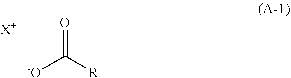

wherein:
   X⁺ is selected from an ammonium cation, an alkyl ammonium cation and a cryptate of an alkali or alkaline earth metal cation; preferably from an ammonium cation or a sodium cryptate of 4,7,13,16,21,24-Hexaoxa-1,10-diazabicyclo [8.8.8]hexacosane (2,2,2-Cryptand, Kryptofix® 222), and more preferably an ammonium cation;
   R is H, a linear or branched C1 to C20 alkyl group; preferably H or methyl and more preferably H.
12. The method according to any one of items 1 to 11, wherein the salt of the alkanoic acid comprises or consists of a formate salt.
13. The method according to item 12, wherein the salt of the alkanoic acid comprises or consists of ammonium formate.
14. The method according to any one of items 1 to 13, wherein the concentration of the salt of the alkanoic acid in the elution composition is in the range of 0.1 to 1.5 mol/l, preferably 0.5 to 1.3 mol/l.
15. The method according to any one of items 1 to 14, wherein the organic solvent comprised by the elution composition comprises or consists of a polar aprotic organic solvent, preferably a solvent selected from dimethyl sulfoxide (DMSO) and acetonitrile (MeCN), and more preferably dimethyl sulfoxide (DMSO).
16. The method according to any one of items 1 to 15, wherein the elution composition or the anion exchange resin has a temperature above room temperature during the step of eluting [¹⁸F]fluoride ions, preferably in the range of 25° C. to less than the boiling point of the organic solvent contained in the elution composition, more preferably in the range of 25° C. to 120° C.
17. The method according to any one of items 1 to 16, wherein the ratio of the volume of the elution composition which is passed through the solid phase extraction device to the mass of the anion exchange resin in the solid phase extraction device in μL/mg is in the range of 2:1 to 40:1, preferably 5:1 to 20:1 and more preferably 5:1 to 15:1.
18. The method according to any one of items 1 to 17, wherein the volume of the elution composition which is passed through the solid phase extraction device is in the range of 100 to 2000 μL, preferably 300 to 1000 μL, more preferably 400 to 600 μL.
19. The method according to any one of items 1 to 18, wherein the water content of the composition obtained as an eluate is in the range of 0 to 5% (vol./vol.), preferably 0 to 2% (vol./vol.), based on the total volume of the eluate composition.
20. The method according to any one of items 1 to 19, wherein each organic solvent used in the method is an anhydrous organic solvent.
21. The method according to any one of items 1 to 20, wherein the composition obtained as an eluate is essentially free of water.
22. The method according to any one of items 1 to 21, which is free from any step wherein water is removed via evaporation.
23. The method according to any one of items 1 to 22, wherein the elution composition further comprises an organic compound to be radiofluorinated.
24. A method for the preparation of a radiofluorinated organic compound, wherein the method comprises the steps of
   preparing a composition by the method in accordance with any of items 1 to 22, which composition comprises an organic solvent, a salt of an alkanoic acid and dissolved [¹⁸F]fluoride ions; and
   contacting an organic compound to be radiofluorinated with the composition to allow the organic compound to undergo a radiofluorination reaction with a [¹⁸F]fluoride ion comprised in the composition.
25. The method according to item 24, wherein the organic compound to be radiofluorinated is contacted with the composition comprising an organic solvent, a salt of an alkanoic acid and dissolved [¹⁸F]fluoride ions by dissolving or dispersing the organic compound in the composition.
26. The method according to any one of items 24 or 25, wherein the composition that is obtained as an eluate in accordance with the method as defined in any of items 1 to 22 is contacted with the organic compound to be radiofluorinated without any modification of or removal of any of the components dissolved in the composition obtained as an eluate.
27. The method according to any one of items 24 to 26, wherein the composition that is obtained as an eluate in accordance with the method as defined in any of items 1 to 22 is diluted in a solvent selected from the group consisting of dimethyl sulfoxide (DMSO), acetonitrile (MeCN) or other polar aprotic solvents prior to contacting it with the organic compound to be radiofluorinated.
28. The method according to any one of items 24 to 26, wherein the composition that is obtained as an eluate in accordance with the method as defined in any of items 1 to 22 is directly contacted with the organic compound to be radiofluorinated without any modification of the composition obtained as an eluate.
29. A method for the preparation of a radiofluorinated organic compound, wherein the method comprises the steps of
   preparing a composition by the method in accordance with item 23, which composition comprises an organic solvent, a salt of an alkanoic acid, dissolved [¹⁸F]fluoride ions and an organic compound to be radiofluorinated; and
   allowing the organic compound to undergo a radiofluorination reaction with a [¹⁸F]fluoride ion comprised in the composition.
30. The method according to any of items 24 to 29, wherein the organic compound to be radiofluorinated comprises a non-radiofluorinated silicon-based fluoride acceptor (SiFA) moiety with a functional group represented by formula (S-1):

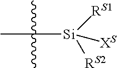

wherein:
X^{S} is ¹⁹F, OH or H, preferably ¹⁹F,
R^{S1} and R^{S2} are independently a linear or branched C3 to C10 alkyl group, preferably R^{S1} and R^{S2} are independently selected from isopropyl and tert-butyl, and more preferably R^{S1} and R^{S2} are tert-butyl, and wherein the waved line marks the bond which attaches the functional group to the remainder of the organic compound to be radiofluorinated;
and wherein the radiofluorination reaction involves an exchange of the group X^{S} by ¹⁸F.
31. The method according to item 30, wherein organic compound to be radiofluorinated comprises a substituted aryl group, which aryl group carries the group of the formula (S-1) as defined in item 30 as a substituent attached to an aromatic ring, and which optionally carries one or more further substituents attached to an aromatic ring in addition to the group of the formula (S-1).
32. The method according to item 31, wherein the substituted aryl group is a substituted phenyl group.
33. The method according to any one of items 24 to 32, wherein the radiofluorination reaction is carried out at a temperature between 10° C. and less than the boiling temperature of the organic solvent contained in the composition comprising an organic solvent, a salt of an alkanoic acid and dissolved [¹⁸F]fluoride ions.
34. The method according to item 33, wherein the radiofluorination reaction is carried out at a temperature between 20° C. and the boiling temperature of the organic solvent contained in the composition comprising an organic solvent, a salt of an alkanoic acid and dissolved [¹⁸F]fluoride ions.
35. The method according to any one of items 24 to 34, further comprising a step of recovering the radiofluorinated organic compound following the radiofluorination reaction.
36. A composition comprising an organic solvent, a salt of an alkanoic acid and dissolved [¹⁸F]fluoride ions.
37. The composition according to item 36, which further comprises an organic compound to be radiofluorinated.
38. The composition in accordance with item 36 or 37, wherein the composition is a composition which is obtainable by the method in accordance with any one of items 1 to 23.
39. The composition in accordance with any of items 36 to 38, wherein the organic solvent comprises a polar aprotic organic solvent selected from dimethyl sulfoxide (DMSO) and acetonitrile (MeCN), and the salt of an alkanoic acid comprises ammonium formate.

In the following, the invention is described in further detail. It will be understood that the information provided in this context also applies for the above items and for the appended claims. The method for the preparation of a composition comprising dissolved [¹⁸F]fluoride ions may be referred to in the following as the method in accordance with the first aspect of the invention, whereas the methods for the preparation of a radiofluorinated organic compound may be referred to in the following as the methods in accordance with the second aspect of the invention.

As an initial step of the method in accordance with the first aspect of the invention, an aqueous solution comprising water and [¹⁸F]fluoride ions is provided. As will be appreciated by the skilled person, ¹⁸F ions for radiopharmaceutical purposes can be generated by irradiation of water containing [¹⁸O]H₂O by protons, e.g. in a cyclotron. In this procedure, a fraction of the [¹⁸O]O²⁻ is converted to [¹⁸F]fluoride ions ([¹⁸F]F⁻). Thus, the aqueous solution comprising water and [¹⁸F]fluoride ions is typically a solution which comprises water as the only solvent.

In order to make the [¹⁸F]fluoride ions available for an efficient radiofluorination reaction, it is desirable to reformulate the aqueous solution obtained from the conversion of [¹⁸O]O²⁻ to [¹⁸F]F⁻ to provide a composition wherein the fluoride ions are dissolved at higher concentrations, and in a solvent which comprises limited amounts of water or which is free of water.

In the method of the first aspect of the present invention, the aqueous solution is passed through a solid phase extraction device comprising an anion exchange resin in order to trap [¹⁸F]fluoride ions on the anion exchange resin and to separate the [¹⁸F]fluoride ions trapped on the anion exchange resin from water. Suitable solid phase extraction devices, such as a solid phase extraction column or a solid phase extraction cartridge, are known to the skilled person and are commercially available. In order to be able to retain [¹⁸F]fluoride ions while allowing water to pass through the device, the solid phase extraction device comprises an anion exchange resin, i.e. a resin which carries positively charged ionic functional groups, preferably quaternary ammonium groups such as —N(CH₃)₃⁺ groups. It is desirable to trap a large portion of the [¹⁸F]fluoride ions contained in the aqueous solution, ideally most of or essentially all of the [¹⁸F]fluoride ions. This can be achieved by adapting the ion exchange capacity of the extraction device to the amount of [¹⁸F]fluoride ions provided in the aqueous solution that is passed through the device.

It will be understood that the separation of the [¹⁸F]fluoride ions trapped on the anion exchange resin from water is accomplished by allowing water to pass through the device while [¹⁸F]fluoride ions are left in the device. Thus, a large portion of the water contained in the aqueous solution can be removed.

If it is desired to further reduce the amount of water associated with the [¹⁸F]fluoride ions trapped on the anion exchange resin (i.e. to further dry the [¹⁸F]fluoride ions), one or more additional steps can be included into the method of the invention prior to the step wherein the [¹⁸F]fluoride ions are eluted from the resin.

For example, the method of the first aspect may further comprise a step (a) of purging the solid phase extraction device comprising the trapped [¹⁸F]fluoride ions on the anion exchange resin with a gas after the aqueous solution has been passed through the device, for example a gas is selected from air, nitrogen, helium and argon, or from mixtures of two or more of these. It will be understood that the gas can be dried before being used for purging.

Another step that may be included into the method of the first aspect of the invention to dry the fluoride prior to the step wherein the fluoride ions are eluted from the resin is a step (b) of rinsing the anion exchange resin comprising the trapped [¹⁸F]fluoride ions with an organic solvent prior to eluting [¹⁸F]fluoride ions from the anion exchange resin. For this step, a single solvent or a mixture of two or more solvents may be used, and a single solvent is preferred. If a mixture of two or more organic solvents is used, it will be understood that the following preferred characteristics are preferred for each solvent of the mixture.

It is preferred that the organic solvent is an anhydrous organic solvent. Preferably, the organic solvent comprises or consists of a polar aprotic solvent, e.g. a solvent selected from acetonitrile (MeCN), dimethyl sulfoxide (DMSO), dimethylacetamide (DMAA), dimethylformamide (DMF) and tetrahydrofuran (THF). More preferably, the organic solvent comprises or consists of a solvent selected from dimethyl sulfoxide (DMSO) and acetonitrile (MeCN), still more preferably comprises or consists of dimethyl sulfoxide (DMSO), and most preferably consists of dimethyl sulfoxide.

If the above step (b) of rinsing the anion exchange resin comprising the trapped [¹⁸F]fluoride ions is included into the method of the invention, it may be followed by a step (c) of purging the solid phase extraction device comprising the trapped [¹⁸F]fluoride ions on the anion exchange resin with a gas after the anion exchange resin has been rinsed with the organic solvent and before the [¹⁸F]fluoride ions are eluted from the resin. Also in this step, an exemplary gas is selected from air, nitrogen, helium and argon, or from mixtures of two or more of these. It will be understood that the gas can be dried before being used for purging.

As will be understood by the skilled reader, the optional additional drying steps may be included as single steps or in a suitable combination into the method in accordance with the invention. For example, the method may comprise, after the step of passing the aqueous solution through the solid phase extraction device, and prior to the step wherein the fluoride ions are eluted from the resin, a step (a), or a step (b), or a step (a) followed by a step (b), or a step (a) followed by a step (b) and a step (c), or a step (b) followed by a step (c), with steps (a), (b) and (c) being defined as above.

After the [¹⁸F]fluoride ions trapped on the anion exchange resin have been separated from water to the desired extent, preferably by removing the water essentially completely or completely, the [¹⁸F]fluoride ions are eluted from the anion exchange resin. In accordance with the invention, this is accomplished using an elution composition comprising an organic solvent and a salt of an alkanoic acid.

The elution composition is generally a liquid composition wherein the salt of the alkanoic acid is dissolved in the organic solvent. Typically, the organic solvent and the salt of the alkanoic acid provide at least 90 wt %, preferably at least 95 wt % of the elution composition, based on the total weight of the elution composition as 100 wt %. The elution composition may consist essentially of the organic solvent and the salt of an alkanoic acid, and more preferably consists of the organic solvent and the salt of an alkanoic acid.

The elution composition may comprise a single organic solvent or a mixture of two or more organic solvents, and a single solvent is preferred. If a mixture of two or more organic solvents is used, it will be understood that the following preferred characteristics are preferred for each solvent of the mixture.

Preferably, the organic solvent comprised by the elution composition comprises or consists of a polar aprotic solvent, e.g. a solvent selected from acetonitrile (MeCN), dimethyl sulfoxide (DMSO), dimethylacetamide (DMAA), dimethylformamide (DMF) and tetrahydrofuran (THF). More preferably, the organic solvent comprised by the elution composition comprises or consists of a solvent selected from dimethyl sulfoxide (DMSO) and acetonitrile (MeCN), still more preferably comprises or consists of dimethyl sulfoxide (DMSO), and most preferably consists of dimethyl sulfoxide.

It is preferred that the organic solvent is an anhydrous organic solvent. Thus, it is also preferred that each organic solvent used in the method in accordance with the invention, e.g. in the optional step of rinsing the anion exchange resin and in the elution composition, is an anhydrous organic solvent.

The elution composition may comprise a single salt of an alkanoic acid or a mixture of two or more of such salts, and a single salt is preferred. If a mixture of two or more salts of alkanoic acids is comprised, it will be understood that the following preferred characteristics are preferred for each salt of the mixture.

The elution composition preferably comprises a salt of an alkanoic acid represented by formula (A-1):

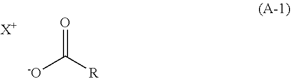

In formula (A-1), X⁺ is selected from an ammonium cation, an alkyl ammonium cation and a cryptate of an alkali or alkaline earth metal cation. It is noted that a cryptate of an alkali or alkaline earth metal cation may be used as a cation for the salt of the alkanoic acid, but that such a cryptate can be absent if another cation, such as an ammonium cation is used. The nitrogen atom of the alkyl ammonium cation may carry one to four alkyl substituents, preferably C1-C6 alkyl substituents, and more preferably methyl substituents. Preferably, X⁺ is selected from an ammonium cation or a sodium or potassium cryptate of 4,7,13,16,21,24-Hexaoxa-1,10-diazabicyclo[8.8.8]hexacosane (2,2,2-Cryptand, Kryptofix® 222), and is more preferably an ammonium cation. R in formula (A-1) is selected from H and a linear or branched C1 to C20 alkyl group; preferably from H and methyl and is more preferably H.

The salt of formula (A-1) preferably provides 90 wt % or more of the salt of an alkanoic acid, more preferably 95 wt % or more, and still more preferably the salt of an alkanoic acid comprised by the elution composition consists of the salt of formula (A-1).

In line with the above, the salt of the alkanoic acid in the elution composition preferably comprises or consist of a formate salt, and more preferably comprises or consist of ammonium formate.

The concentration of the salt of the alkanoic acid in the elution composition is preferably selected such that the salt can be fully dissolved in the organic solvent of the elution composition. To the extent that this limit is not exceeded, higher concentrations are generally more favorable. For example, the concentration of the alkanoic acid in the elution composition can be in the range of 0.1 to 1.5 mol/l, preferably in the range of 0.5 to 1.3 mol/l.

As noted above, the elution composition may comprise an organic compound to be radiofluorinated as an optional further component. As will be understood by the skilled reader, in order to implement this option, the organic compound to be radiofluorinated can be added to the elution composition prior to the step of eluting the [¹⁸F]fluoride ions from the anion exchange resin.

Further in line with the above, it will be understood that an elution composition is particularly preferred which comprises or consists of MeCN or DMSO as organic solvent, a formate salt as a salt of an alkanoic acid, and, as an optional further component, an organic compound to be radiofluorinated, and that still more preferred is an elution composition which comprises or consists of DMSO as organic solvent, ammonium formate as a salt of an alkanoic acid, and, as an optional further component, an organic compound to be radiofluorinated.

The temperature of the elution composition and the anion exchange resin for the step of eluting the [¹⁸F]fluoride ions from the anion exchange resin by passing the elution composition through the solid phase extraction device is not particularly restricted. For example, the elution composition and the anion exchange resin may have a temperature around room temperature (e.g. 15 to 25° C.). If desired to increase the mobility of the [¹⁸F]fluoride ions during the eluting step, the elution composition and the anion exchange resin may have a temperature in the range of 25° C. to less than the boiling point of the organic solvent contained in the elution composition, such as in the range of 25° C. to 120° C. If the elution composition contains more than one organic solvent, it will be understood that the upper limit is generally determined by the organic solvent with the lower boiling point.

The volume of the elution composition which is passed through the solid phase extraction device is not particularly limited, but in order to obtain an eluate with a high concentration of [¹⁸F]fluoride ions, it is favorable to use a volume which is not larger than necessary to elute the major amount of the [¹⁸F]fluoride ions trapped on the anion exchange resin.

In the method in accordance with the first aspect of the invention, the ratio of the volume of the elution composition which is passed through the solid phase extraction device to the mass of the anion exchange resin in the solid phase extraction device is not particularly limited. It can be conveniently adjusted to desired ranges. For example, the ratio of the volume of the elution composition which is passed through the solid phase extraction device (expressed in μL) to the mass of the anion exchange resin in the solid phase extraction device (expressed in mg) can be in the range of 2:1 to 40:1, preferably 5:1 to 20:1 and more preferably 5:1 to 15:1.

For example, the volume of the elution composition which is passed through the solid phase extraction device can be in the range of 100 to 2000 μL, preferably 300 to 1000 μL, and more preferably 400 to 600 μL.

It may be an advantage for the efficiency of the elution step if the flow direction of the eluting composition in this step through the solid phase extraction device is inversed compared to the flow direction of the aqueous solution comprising water and [¹⁸F]fluoride ions in the step in which the fluoride ions are trapped on the anion exchange resin.

In line with the method of the first aspect of the invention, a composition is obtained as an eluate which comprises the organic solvent, the salt of the alkanoic acid, dissolved [¹⁸F]fluoride ions and, as an optional further component, an organic compound to be radiofluorinated. This composition, which can be obtained as a product by the method in accordance with the first aspect of the invention, forms a further aspect of the present invention. The composition which is prepared by the method in accordance with the first aspect of the invention and which is obtained as the eluate in this method may be referred to in the following as “eluate composition” or simply as “eluate”.

As will be understood by the skilled reader, the contents of the eluate composition are generally determined by the elution composition which is used in the method in accordance with the first aspect of the invention. Therefore, the information provided above with respect to the organic solvent and the salt of an alkanoic acid of the eluting composition continues to apply for the organic solvent and the salt of an alkanoic acid of the eluate composition, except for the fact that a fraction of the alkanoate anions of the eluting composition is replaced in the eluate composition by the eluted [¹⁸F]fluoride ions.

Thus, the eluate composition is generally a liquid composition wherein the salt of the alkanoic acid and the [¹⁸F]fluoride ions are dissolved in the organic solvent. The organic solvent, the salt of the alkanoic acid and the dissolved [¹⁸F]fluoride ions may provide at least 90 wt %, preferably at least 95 wt % of the eluate composition, based on the total weight of the eluate composition as 100 wt %. The eluate composition may consist essentially of the organic solvent, the salt of an alkanoic acid, and the dissolved [¹⁸F]fluoride ions, and more preferably consists of the organic solvent, the salt of an alkanoic acid, and the dissolved [¹⁸F]fluoride ions.

In accordance with an alternative embodiment, the eluate composition may comprise, as an optional further component, an organic compound to be radiofluorinated or an organic compound to be radiofluorinated and a radiofluorinated compound, since the radiofluorination reaction may proceed to a certain extent during the step of eluting the [¹⁸F]fluoride ions in the presence of an organic compound to be radiofluorinated.

The eluate composition may comprise a single organic solvent or a mixture of two or more organic solvents, and a single solvent is preferred. If a mixture of two or more organic solvents is used, it will be understood that the following preferred characteristics are preferred for each solvent of the mixture.

Preferably, the organic solvent comprised by the eluate composition comprises or consists of a polar aprotic solvent, e.g. a solvent selected from acetonitrile (MeCN), dimethyl sulfoxide (DMSO), dimethylacetamide (DMAA), dimethylformamide (DMF) and tetrahydrofuran (THF). More preferably, the organic solvent comprised by the eluate composition comprises or consists of a solvent selected from dimethyl sulfoxide (DMSO) and acetonitrile (MeCN), still more preferably comprises or consists of dimethyl sulfoxide (DMSO), and most preferably consists of dimethyl sulfoxide.

It is preferred that the organic solvent is an anhydrous organic solvent.

The eluate composition may comprise a single salt of an alkanoic acid or a mixture of two or more of such salts, and a single salt is preferred. If a mixture of two or more salts of alkanoic acids is comprised, it will be understood that the following preferred characteristics are preferred for each salts of the mixture.

The eluate composition preferably comprises a salt of an alkanoic acid represented by formula (A-1):

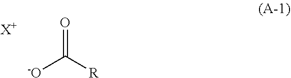

In formula (A-1), X⁺ is selected from an ammonium cation, an alkyl ammonium cation and a cryptate of an alkali or alkaline earth metal cation. It is noted that a cryptate of an alkali or alkaline earth metal cation may be used as a cation for the salt of the alkanoic acid, but that such a cryptate can be absent if another cation, such as an ammonium cation is used. The nitrogen atom of the alkyl ammonium cation may carry one to four alkyl substituents, preferably C1-C6 alkyl substituents, and more preferably methyl substituents. Preferably, X⁺ is selected from an ammonium cation or a sodium or potassium cryptate of 4,7,13,16,21,24-Hexaoxa-1,10-diazabicyclo[8.8.8]hexacosane (2,2,2-Cryptand, Kryptofix® 222), and is more preferably an ammonium cation. R in formula (A-1) is selected from H and a linear or branched C1 to C20 alkyl group; preferably from H and methyl and is more preferably H.

The salt of formula (A-1) preferably provides 90 wt % or more of the salt of an alkanoic acid, more preferably 95 wt % or more, and still more preferably the salt of an alkanoic acid comprised by the eluate composition consists of the salt of formula (A-1).

In line with the above, the salt of the alkanoic acid in the eluate composition preferably comprises or consist of a formate salt, and more preferably comprises or consist of ammonium formate.

The concentration of the salt of the alkanoic acid in the eluate composition is, for example, in the range of 0.1 to 1.5 mol/l, preferably in the range of 0.5 to 1.3 mol/l. Due to the relatively small concentration of the dissolved [¹⁸F]fluoride ions, the concentration of the salt is typically not significantly changed when the fluoride ions are eluted.

Further in line with the above, it will be understood that an eluate composition is particularly preferred which comprises or consists of MeCN or DMSO as organic solvent, a formate salt as a salt of an alkanoic acid, the dissolved [¹⁸F]fluoride ions, and, as an optional further component, an organic compound to be radiofluorinated or an organic compound to be radiofluorinated and a radiofluorinated organic compound. Still more preferred is an eluate composition which comprises or consists of DMSO as organic solvent, ammonium formate as a salt of an alkanoic acid, the dissolved [¹⁸F]fluoride ions, and, as an optional further component, an organic compound to be radiofluorinated or an organic compound to be radiofluorinated and a radiofluorinated organic compound.

Using the method in accordance with the invention, the concentration of the [¹⁸F]fluoride ions in the eluate composition can be conveniently adjusted according to need. For example, the concentration of the [¹⁸F]fluoride ions may be in the range of 10 MBq to 150 GBq, indicated for a 500 μL volume of the eluate composition.

The water content of the eluate composition is preferably in the range of 0 to 5% (vol./vol.), more preferably 0 to 2% (vol./vol.), based on the total volume of the eluate composition. It is still more preferred that the eluate composition is essentially free, even more preferably free, of water.

Due to the possibility of drying the trapped [¹⁸F]fluoride ions, and/or of using anhydrous solvents while still ensuring an efficient recovery of the [¹⁸F]fluoride ions from the anion exchange resin, the method in accordance with the first aspect of the invention as discussed above can be free from any step wherein water is removed via evaporation, and such a step is not needed prior to or during the use of the eluate composition in a radiofluorination reaction either.

As will be understood from the above, the method in accordance with the first aspect of the invention can be advantageously used e.g. to extract [¹⁸F]fluoride ions from an aqueous solution, to concentrate the [¹⁸F]fluoride ions and/or to reformulate the [¹⁸F]fluoride ions.

The method for the preparation of a radiofluorinated organic compound in accordance with the second aspect of the invention comprises the steps of
preparing a composition comprising an organic solvent, a salt of an alkanoic acid and dissolved [¹⁸F]fluoride ions in accordance with the method of the first aspect of the invention discussed above; and
contacting the organic compound to be radiofluorinated with the composition to allow the organic compound to undergo a radiofluorination reaction with a [¹⁸F]fluoride ion comprised in the composition. As the product of the radiofluorination reaction, a radiofluorinated organic compound is obtained.

It will be understood that the information which is provided above with respect to details and preferred embodiments of the method of the first aspect of the invention discussed above fully applies for the method in accordance with the second aspect of the invention, which includes the preparation of a composition comprising an organic solvent, a salt of an alkanoic acid and dissolved [¹⁸F]fluoride ions in accordance with the method of the first aspect of the invention.

Generally, as noted above, it is an advantage of the method in accordance with the first aspect of the invention that the obtained eluate composition is immediately applicable for a subsequent radiofluorination reaction as it is carried out in the method of the second aspect of the invention. For example, it is not necessary that an acid is added to the eluate composition in order to adjust the pH value of the composition prior to contacting the composition with a compound to be subjected to a radiofluorination reaction. For example, it is possible to contact the eluate composition that is obtained in the process in accordance with the first aspect of the invention without subjecting it to any further processing steps, with an organic compound to be radiofluorinated.

Thus, it is a preferred variant of the method in accordance with the second aspect of the invention that the composition obtained as an eluate in accordance with the method of the first aspect is contacted with the organic compound to be radiofluorinated without any modification of or removal of any of the components dissolved in the composition obtained as an eluate.

In a more preferred variant, the organic solution that is obtained as an eluate in accordance with the method of the first aspect is directly contacted with the organic compound to be radiofluorinated, without any modification of the eluate composition.

However, if desired the composition that is obtained as an eluate in accordance with the method of the first aspect can e.g. be diluted in a solvent, preferably a polar aprotic solvent, prior to contacting it with the organic compound to be radiofluorinated. Exemplary solvents are selected from the group consisting of dimethyl sulfoxide (DMSO) and acetonitrile (MeCN).

Typically, the organic compound to be radiofluorinated is contacted with the composition comprising an organic solvent, a salt of an alkanoic acid and dissolved [¹⁸F]fluoride ions by dissolving or dispersing the organic compound in the composition.

However, as a variant of the method for the preparation of a radiofluorinated organic compound in accordance with the second aspect, the organic compound to be radiofluorinated can also be added to the elution composition comprising an organic solvent and a salt of an alkanoic acid. In accordance with this variant, a method for the preparation of a radiofluorinated organic compound is provided wherein the organic compound to be radiofluorinated no longer needs to be contacted with the composition prepared in accordance with the first aspect of the invention. Rather, this method for the preparation of a radiofluorinated organic compound comprises the steps of
preparing a composition comprising an organic solvent, a salt of an alkanoic acid, dissolved [¹⁸F]fluoride ions and an organic compound to be radiofluorinated in accordance with the embodiment of the method of the first aspect of the invention wherein the elution composition further comprises an organic compound to be radiofluorinated; and
allowing the organic compound to undergo a radiofluorination reaction with a [¹⁸F]fluoride ion comprised in the composition.

As will be understood by the skilled reader, a radiofluorinated organic compound as referred to herein is an organic compound to which a radioactive [¹⁸F]fluorine atom is attached by a chemical bond, typically by a covalent bond. Thus, radiofluorination (or a radiofluorination reaction) refers to a step wherein the organic compound reacts to form a chemical bond, typically a covalent bond, with a radioactive [¹⁸F]fluorine atom. In the method according to the second aspect of the present invention, the radiofluorination or radiofluorination reaction is accomplished by reacting the organic compound with a [¹⁸F]fluoride ion.

The organic compound to be radiofluorinated preferably comprises a non-radiofluorinated silicon-based fluoride acceptor (SiFA) moiety, i.e. a group wherein a silicon atom carries an atom or group which is covalently bound to the silicon atom and which can be replaced by ¹⁸F in the radiofluorination reaction. Preferably, the SiFA moiety provides a functional group represented by formula (S-1):

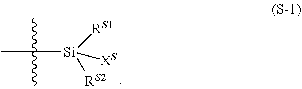

In formula (S-1), the group X^{S} attached to the Si atom is ¹⁹F, OH or H, preferably ¹⁹F. R^{S1} and R^{S2} are independently a linear or branched C3 to C10 alkyl group, preferably R^{S1} and R^{S2} are independently selected from isopropyl and tert-butyl, and more preferably R^{S1} and R^{S2} are tert-butyl. Thus, it will be understood that a particularly preferred SiFA moiety in an organic compound to be radiofluorinated has a functional group of formula (S-1) wherein X^{S} is ¹⁹F and R^{S1} and R^{S2} both tert-butyl. The waved line in formula (S-1) marks the bond which attaches the functional group to the remainder of the organic compound.

Preferably, the organic compound to be radiofluorinated comprises a substituted aryl group, which aryl group carries a group of the formula (S-1) as a substituent attached to an aromatic ring, and which optionally carries one or more, such as one, two, or three, further substituents attached to an aromatic ring in addition to the group of the formula (S-1). More preferably, the organic compound to be radiofluorinated comprises a substituted phenyl group, which phenyl carries a group of the formula (S-1) as a substituent attached to the phenyl ring, and which optionally carries one or more, such as one, two, or three, further substituents attached to the phenyl ring in addition to the group of the formula (S-1).

If the organic compound to be radiofluorinated comprises a non-radiofluorinated silicon-based fluoride acceptor (SiFA) moiety with a functional group represented by formula (S-1), the radiofluorination reaction of the organic compound involves an exchange of the group X^{S} by ¹⁸F.

It is further preferred that the SiFA moiety is a group of the formula (S-2):

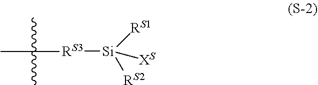

wherein X^{S}, R^{S1} and R^{S2} are defined as for (S-1) above, including their preferred embodiments, and R^{S3} is a divalent C1 to C20 hydrocarbon group which comprises one or more aromatic and/or aliphatic moieties, and which optionally carries one or more, such as one, two, or three further substituents in addition to the substituents of R^{S3} shown in formula (S-2). Such optional substituents can be, e.g., organic functional groups. Preferably, R^{S3} is a divalent C6 to C12 hydrocarbon group which comprises an aromatic ring and which may comprise one or more aliphatic moieties, and which optionally carries one or more, such as one, two, or three further substituents in addition to the substituents of R^{S3} shown in formula (S-2). Such optional substituents can be, e.g., organic functional groups, and, if present, are preferably attached to the aromatic ring. The waved line in formula (S-2) marks the bond which attaches the functional group to the remainder of the organic compound. The radiofluorination reaction of the organic compound comprising the group (S-2) also involves an exchange of the group X^{S} by ¹⁸F.

Still more preferred as a SiFA moiety in the compound to be radiofluorinated is a group of the formula (S-3):

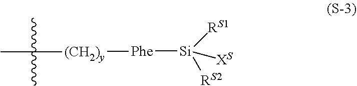

wherein R^{S1} and R^{S2} are defined as for (S-1) above, including their preferred embodiments, F is a ¹⁹F atom which is replaced by ¹⁸F during the radiofluorination reaction, Phe is a phenylene group which optionally carries one or more, such as one, two, or three further substituents in addition to the substituents of Phe shown in formula (S-3). Such optional substituents can be, e.g., organic functional groups. y is an integer of 0 to 6, preferably 0 or 1. The waved line marks a bond which attaches the group to the remainder of the compound. The two substituents shown in formula (S-3) on the phenylene group (i.e. the group (CH₂)_{y} and the Si-containing group) are preferably in para-position to each other. It is particularly preferred that the compound to be radiofluorinated comprises a group of formula (S-3) wherein R^{S1} and R^{S2} are tert-butyl, wherein y is 0 or 1, and wherein the two substituents shown in formula (S-3) on the phenylene group are in para-position to each other.

Suitable organic functional groups which may be present as optional substituents in the groups of formula (S-2) and (S-3) are, e.g., groups comprising one, two or three heteroatoms selected from O, N and S, and a total of 6 atoms including the heteroatoms, C and H.

The radiofluorination reaction is typically carried out at a temperature between 10° C., more preferably 20° C., and less than the boiling temperature of the organic solvent contained in the composition comprising an organic solvent, a salt of an alkanoic acid and dissolved [¹⁸F]fluoride ions. If the composition contains more than one organic solvent, it will be understood that the upper limit is generally determined by the organic solvent with the lower boiling point. For example, a suitable temperature range may be 10° C., more preferably 20° C., to 150° C.

If desired in order to accelerate the reaction, the radiofluorination reaction can be carried out at temperatures above room temperature, such as 50° C. or more, 70° C. or more or 90° C. or more. As explained above, it is an advantage of the eluate composition provided in the context of the invention that it does not affect the structural integrity of organic compounds to be radiofluorinated even at increased temperatures.

As will be understood, the method according to the second aspect may also comprise a step of recovering the radiofluorinated organic compound following the radiofluorination reaction.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### REFERENCES

### Patent Documents

P1. H.-J. Wester, G. Henriksen, S. Weßmann, Method for the direct elution of reactive [18F]fluoride from an anion exchange resin in an organic medium suitable for radiolabelling without any evaporation step by the use of alkalimetal and alkaline earth metal cryptates, WO 2011/141410.
P2. A. Wurzer, H.-J. Wester, M. Eiber, PSMA binding dual mode radiotracer and therapeutic, WO 2020/157177 A1.
P3. D. Di Carlo, H.-J. Wester, Silicon-fluoride acceptor substituted radiopharmaceuticals and precursors thereof, WO 2020/157128 A1.

### Non-Patent Literature

1. Schirrmacher, R.; Wängler, C.; Schirrmacher, E., Fluorine-18 Radiochemistry: Theroy and Practice. Pharmaceutical Radiochemistry (I) 2010, 1, 5-73.
2. Tredwell, M.; Gouverneur, V., 18F labeling of arenes. Angewandte Chemie International Edition 2012, 51 (46), 11426-11437.
3. Jadvar, H.; Parker, J. A., Clinical PET and PET/CT. Springer Science & Business Media: 2006.
4. Bernard-Gauthier, V.; Bailey, J. J.; Liu, Z. B.; Wangler, B.; Wangler, C.; Jurkschat, K.;
Perrin, D. M.; Schirrmacher, R., From Unorthodox to Established: The Current Status of F-18-Trifluoroborate- and F-18-SiFA-Based Radiopharmaceuticals in PET Nuclear Imaging. Bioconjugate Chemistry 2016, 27 (2), 267-279.
5. Bernard-Gauthier, V.; Wängler, C.; Schirrmacher, E.; Kostikov, A.; Jurkschat, K.; Wängler, B.; Schirrmacher, R., 18F-Labeled silicon-based fluoride acceptors: Potential opportunities for novel positron emitting radiopharmaceuticals. BioMed research international 2014, 2014.
6. Schirrmacher, R.; Bradtmöller, G.; Schirrmacher, E.; Thews, O.; Tillmanns, J.; Siessmeier, T.; Buchholz, H. G.; Bartenstein, P.; Wängler, B.; Niemeyer, C. M., 18F-Labeling of Peptides by means of an Organosilicon-Based Fluoride Acceptor. Angewandte Chemie International Edition 2006, 45 (36), 6047-6050.
7. Ting, R.; Adam, M. J.; Ruth, T. J.; Perrin, D. M., Arylfluoroborates and alkylfluorosilicates as potential PET imaging agents: high-yielding aqueous biomolecular 18F-labeling. Journal of the American Chemical Society 2005, 127 (38), 13094-13095.
8. Wängler, C.; Niedermoser, S.; Chin, J.; Orchowski, K.; Schirrmacher, E.; Jurkschat, K.; Iovkova-Berends, L.; Kostikov, A. P.; Schirrmacher, R.; Wängler, B., One-step 18 F-labeling of peptides for positron emission tomography imaging using the SiFA methodology. nature protocols 2012, 7 (11), 1946-1955.
9. Bernard-Gauthier, V.; Bailey, J. J.; Liu, Z.; Wängler, B. r.; Wängler, C.; Jurkschat, K.; Perrin, D. M.; Schirrmacher, R., From unorthodox to established: The current status of 18F-trifluoroborate- and 18F-SiFA-based radiopharmaceuticals in PET nuclear imaging. Bioconjugate chemistry 2015, 27 (2), 267-279.
10. Wessmann, S.; Henriksen, G.; Wester, H.-J., Cryptate mediated nucleophilic 18F-fluorination without azeotropic drying. Nuklearmedizin 2012, 51 (01), 1-8.
11. Wurzer, A.; Di Carlo, D.; Schmidt, A.; Beck, R.; Schwaiger, M.; Herz, M.; Eiber, M.;
Wester, H., PSMA-targeted 18F-labeled Radiohybrid Inhibitors: Labeling chemistry and automated GMP production of 18F-rhPSMA-7. Journal of Nuclear Medicine 2019, 60 (supplement 1), 342-342.
12. Wurzer, A.; Di Carlo, D.; Schmidt, A.; Beck, R.; Eiber, M.; Schwaiger, M.; Wester, H.-J., Radiohybrid Ligands: A Novel Tracer Concept Exemplified by 18F- or 68Ga-Labeled rhPSMA Inhibitors. Journal of Nuclear Medicine 2020, 61 (5), 735-742.
13. Kuntzsch, M.; Lamparter, D.; Brüggener, N.; Müller, M.; Kienzle, G. J.; Reischl, G., Development and successful validation of simple and fast TLC spot tests for determination of Kryptofix® 2.2.2 and tetrabutylammonium in 18F-labeled radiopharmaceuticals. Pharmaceuticals 2014, 7 (5), 621-633.
14. Brichard, L.; Aigbirhio, F. I., An Efficient method for enhancing the reactivity and flexibility of [18F]fluoride towards nucleophilic substitution using tetraethylammonium bicarbonate. European Journal of Organic Chemistry 2014, 2014 (28), 6145-6149.
15. Inkster, J.; Akurathi, V.; Sromek, A.; Chen, Y.; Neumeyer, J.; Packard, A., A non-anhydrous, minimally basic protocol for the simplification of nucleophilic 18 F-fluorination chemistry. Scientific Reports 2020, 10 (1), 1-9.
16. Wurzer, A.; Di Carlo, D.; Herz, M.; Richter, A.; Robu, S.; Schirrmacher, R.; Mascarin, A.; Weber, W.; Eiber, M.; Schwaiger, M. and Wester H.-J., Automated Synthesis of [18F, natGa]rhPSMA-7/-7.3: Results, Quality Control and Experience from more than 200 Routine Productions. EJNMMI Radiopharmacy and Chemistry, submitted.
The following examples serve to illustrate the invention.

### EXAMPLES

### Materials

Aq. [¹⁸F]fluoride (approx. 0.6-2.0 GBq/mL) for radiofluorination was provided by the Klinikum rechts der Isar (Munich, Germany) and produced in the on-site PETtrace™ 880 cyclotron (GE Healthcare GmbH, Solingen, Germany). A CRC®-55tR dose calibrator from Capintec Inc. (Florham Park, NJ, United States) was used for activity measurements.

Sep-Pak® Accell Plus QMA Carbonate Plus Light cartridge (46 mg sorbent weight, 40 μm particle size, 230 μeqg⁻¹ ion exchange capacity) for preparation of [¹⁸F]fluoride and Oasis® HLB Plus Light cartridge (30 mg sorbent weight, 30 μm particle size) for purification of ¹⁸F-labeled compounds were supplied by Waters GmbH (Eschborn, Germany).

NBu₄OTf, NBuI, NH₄I, NH₄OAc, NH₄HCOO, KOH (quality grade “99.99%, semiconductor grade”), oxalic acid (quality grade “99.999% trace metals basis”) and anhyd. DMSO (quality grade “≥99.9%”) were purchased from Sigma-Aldrich Chemie GmbH (Steinheim, Germany). NMe₄OAc was supplied by TCI Deutschland GmbH (Eschborn, Germany). Kryptofix® 222 (quality grade “for synthesis”), water (quality grade “Tracepur®”) and abs. EtOH (quality grade “EMPARTA®”) were provided by Merck KGaA (Darmstadt, Germany). Anhyd. MeCN (quality grade “≥99.9% for DNA synthesis”) was purchased from VWR International GmbH (Darmstadt, Germany). Further reagents, solvents and buffers were delivered by either Sigma-Aldrich Chemie GmbH or Merck KGaA.

Ligand precursors used for radiofluorination as shown in the following were synthesized according to the procedures reported in the literature.^{P2-P3}

Chemical structure of ^{nat}Ga-rhPSMA-7.3.

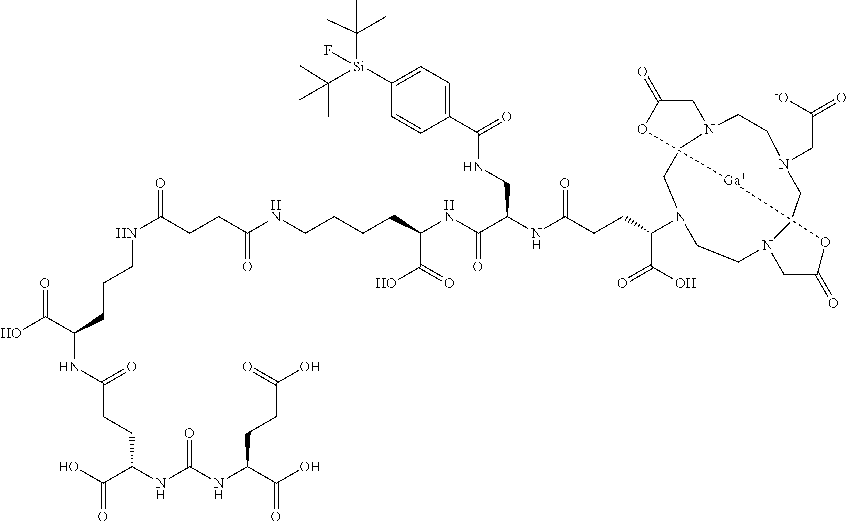

^{nat}Ga-rhPSMA-7.3
Chemical Formula: C₆₃H₉₆FGaN₁₂O₂₅Si
Molecular Weight: 1538.33 g/mol

Chemical structures of siPSMA-01 to-09.

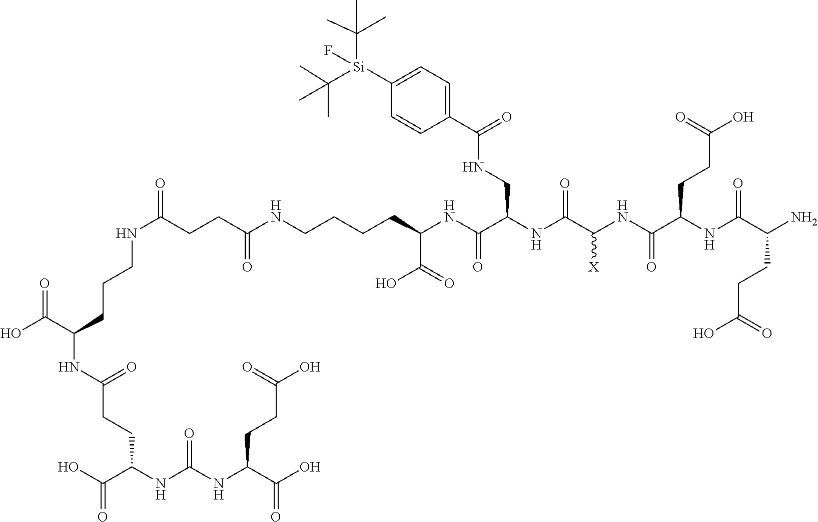

siPSMA-01 to-09
siPSMA-01
X = CH₂COOH: D-Asp
Chemical Formula: C₅₈H₈₈FN₁₁O₂₅Si
Molecular Weight: 1386.48 g/mol
siPSMA-02
X = (CH₂)₂COOH: D-Glu
Chemical Formula: C₅₉H₉₀FN₁₁O₂₅Si
Molecular Weight: 1400.50 g/mol
siPSMA-03
X = CH(OH)CH₃: D-Thr
Chemical Formula: C₅₈H₉₀FN₁₁O₂₄Si
Molecular Weight: 1372.49 g/mol
siPSMA-04
X = (CH₂)₃NHCONH₂: D-Cit
Chemical Formula: C₆₀H₉₄FN₁₃O₂₄Si
Molecular Weight: 1428.56 g/mol
siPSMA-05
X = CH₂C₆H₅: D-Phe
Chemical Formula: C₆₃H₉₂FN₁₁O₂₃Si
Molecular Weight: 1418.57 g/mol
siPSMA-06
X = CH₂NH₂: D-Dap
Chemical Formula: C₅₇H₈₉FN₁₂O₂₃Si
Molecular Weight: 1357.48 g/mol
siPSMA-07
X = (CH₂)₃NH₂: D-Orn
Chemical Formula: C₅₉H₉₃FN₁₂O₂₃Si
Molecular Weight: 1385.54 g/mol
siPSMA-08
X = (CH₂)₄NH₂: D-Lys
Chemical Formula: C₆₀H₉₅FN₁₂O₂₃Si
Molecular Weight: 1399.56 g/mol
siPSMA-09
X = H: Gly
Chemical Formula: C₅₆H₈₆FN₁₁O₂₃Si
Molecular Weight: 1328.44 g/mol

Chemical structures of siPSMA-11 to-18L.

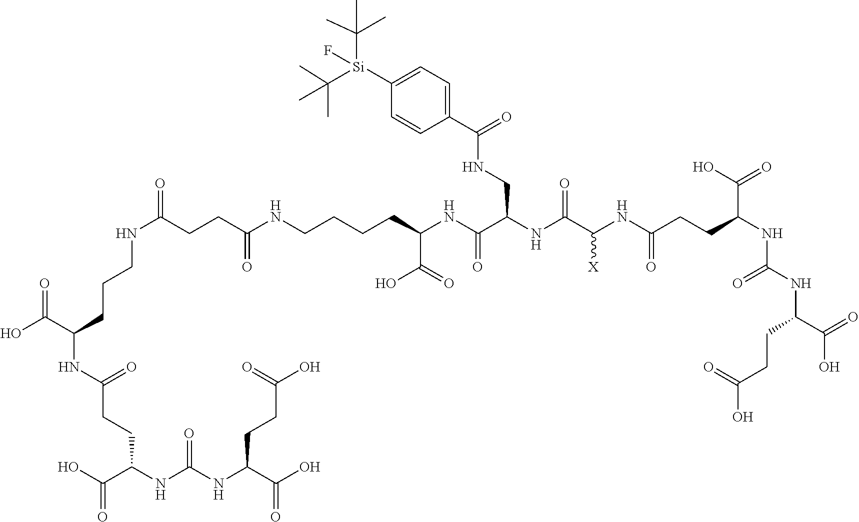

siPSMA-11 to-18L
siPSMA-11
X = CH₂COOH: D-Asp
Chemical Formula: C₅₉H₈₈FN₁₁O₂₇Si
Molecular Weight: 1430.49 g/mol
siPSMA-12D
X = (CH₂)₂COOH: D-Glu
Chemical Formula: C₆₀H₉₀FN₁₁O₂₇Si
Molecular Weight: 1444.51 g/mol
siPSMA-12L
X = (CH₂)₂COOH: L-Glu
Chemical Formula: C₆₀H₉₀FN₁₁O₂₇Si
Molecular Weight: 1444.51 g/mol
siPSMA-13
X = CH(OH)CH₃: D-Thr
Chemical Formula: C₅₉H₉₀FN₁₁O₂₆Si
Molecular Weight: 1416.50 g/mol
siPSMA-14
X = (CH₂)₃NHCONH₂: D-Cit
Chemical Formula: C₆₁H₉₄FN₁₃O₂₆Si
Molecular Weight: 1472.57 g/mol
siPSMA-15
X = CH₂C₆H₅: D-Phe
Chemical Formula: C₆₄H₉₂FN₁₁O₂₅Si
Molecular Weight: 1462.58 g/mol
siPSMA-16
X = CH₂NH₂: D-Dap
Chemical Formula: C₅₈H₈₉FN₁₂O₂₅Si
Molecular Weight: 1401.49 g/mol
siPSMA-17
X = (CH₂)₃NH₂: D-Orn
Chemical Formula: C₆₀H₉₃FN₁₂O₂₅Si
Molecular Weight: 1429.55 g/mol
siPSMA-18D
X = (CH₂)₄NH₂: D-Lys
Chemical Formula: C₆₁H₉₅FN₁₂O₂₅Si
Molecular Weight: 1443.57 g/mol
siPSMA-18L
X = (CH₂)₄NH₂: L-Lys
Chemical Formula: C₆₁H₉₅FN₁₂O₂₅Si
Molecular Weight: 1443.57 g/mol

Chemical structures of siPSMA-19 to-21.

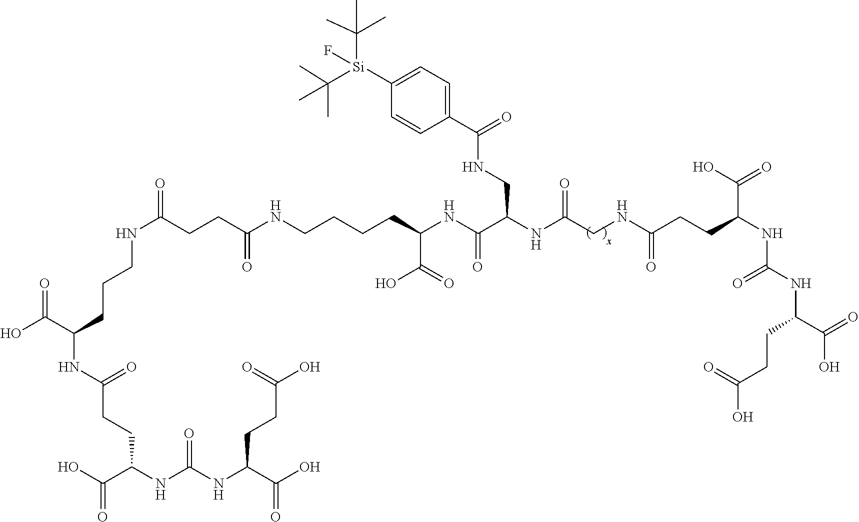

siPSMA-19 to-21
siPSMA-19
x = 1: Gly
Chemical Formula: C₅₇H₈₆FN₁₁O₂₅Si
Molecular Weight: 1372.45 g/mol
siPSMA-20
x = 2: b-Ala
Chemical Formula: C₅₈H₈₈FN₁₁O₂₅Si
Molecular Weight: 1386.48 g/mol
siPSMA-21
x = 5: Ahx
Chemical Formula: C₆₁H₉₄FN₁₁O₂₅Si
Molecular Weight: 1428.56 g/mol

Analytical characterization of ¹⁸F-labeled compounds was performed on column I (MultoKrom® 100-5 C18, 125× 4.6 mm, 5 μm, 1 mL/min, CS-Chromatographie Service GmbH, Langerwehe, Germany) or column II (MultoKrom® 100-5 C18, 150× 4.6 mm, 5 μm, 1 mL/min, CS-Chromatographie Service GmbH) in an HPLC system (Shimadzu Deutschland GmbH, Neufahrn bei Freising, Germany) consisting of gradient pumps (two LC-20AD), an autosampler (SIL-20AHT), a system controller (CBM-20A), a column oven (CTO-10ASVP), an UV/Vis detector (SPD-20A) and a LB 500 HERM radio flow monitor with NaI detector (Berthold Technologies Gmbh & Co. KG, Bad Wildbad, Germany). Radiolabeled compounds were eluted applying different gradients of solvent A (water, add. 0.1% TFA, v/v) and solvent B (MeCN, add. 0.1% TFA, add. 2% water, v/v/v) at a constant flow. LabSolutions 5.92 software by Shimadzu Deutschland GmbH was employed for analysis of radiochromatograms.

### Methods

### General Procedures for [¹⁸F]Fluoride Preparation

GP1: Aq. [¹⁸F]fluoride was trapped (male side) onto the QMA cartridge previously precon, with water (10 mL). After drying with air (2×20 mL, female side), the cartridge was slowly rinsed with anhyd. DMSO (8 mL, female side) and subsequently dried with air (2× 20 mL, female side) again.
GP2: Aq. [¹⁸F]fluoride was trapped (male side) onto the QMA cartridge previously precon. with water (10 mL). After drying with air (2×20 mL, female side), the cartridge was slowly rinsed with anhyd. MeCN (10 mL, female side) and subsequently dried with air (2× 20 mL, female side) again.

### General Procedures for [¹⁸F]Fluoride Elution

GE1: Dried [¹⁸F]fluoride was eluted (female side) from the QMA cartridge with an elution cocktail composed of NH₄HCOO (40 mg, 634 μmol) in anhyd. DMSO (500 μL). The QMA cartridge was subsequently rinsed with air (20 mL, female side) and the resulting droplets were unified with the previous eluate.
GE2: (Reference procedure for comparative purposes) Dried [¹⁸F]fluoride was eluted (female side) from the QMA cartridge with an elution cocktail composed of KOH (4.7 mg, 83 μmol) and Kryptofix® 222 (34 mg, 91 μmol) in anhyd. MeCN (500 μL). The QMA cartridge was subsequently rinsed with air (20 mL, female side) and the resulting droplets were unified with the previous eluate. The eluate was thereafter partly neutralized with a solution (1 M, 30 μL, 30 μmol) of oxalic acid in anhyd. MeCN.

### General Procedures for Radiofluorination

GR1: The [18F]fluoride eluate was incubated with a solution (1 mm, 150 μL, 150 nmol) of the precursor compound in anhyd. DMSO for 5 min at rt.
GR2: The [18F]fluoride eluate was incubated with a solution (1 mm, 30 μL, 30 nmol) of the precursor compound in anhyd. DMSO for 5 min at rt.
GR3: The [18F]fluoride eluate was incubated with a solution (1 mM, 30 μL, 30 nmol) of the precursor compound in anhyd. DMSO for 10 min at rt.
GR4: The [18F]fluoride eluate was incubated with a solution (1 mm, 30 μL, 30 nmol) of the precursor compound in anhyd. DMSO for 10 min at 95° C.

GR5: The [18F]fluoride eluate was incubated with a solution (1 mm, 0.5 L, 0.5 nmol) of the precursor compound in anhyd. DMSO for 8 min at 65° C.

GR6: The [¹⁸F]fluoride eluate was incubated with a solution (1 mm, 0.5 μL, 0.5 nmol) of the precursor compound in anhyd. DMSO for 5 min at 70° C.

### General Procedure for Work-Up of ¹⁸F-Labeled Compounds

GW1: The reaction mixture was diluted with PBS (pH=3 with 1 M aq. HCl, 10 mL) and passed (female side) through the HLB cartridge previously precon. with abs. EtOH (10 mL) and water (10 mL). Finally, the HLB cartridge was rinsed with PBS (10 mL, female side), dried with air (20 mL, female side) and the radiofluorinated compound was eluted with a mixture (1:1, v/v, 300 μL, female side) of abs. EtOH and water.

### Development of the Invention

As the on-cartridge drying method of [¹⁸F]fluoride demonstrated to be more convenient compared to the classic azeotropic distillation in terms of ease and efficiency, this approach was integrated in the present invention. Thus, aq. [¹⁸F]fluoride was trapped on the Sep-Pak® QMA Carbonate (46 mg sorbent weight, 230 μeqg⁻¹ ion exchange capacity), dried with air, MeCN (10 mL) and air again before being inversely eluted. The first challenge consisted in finding an alternative elution cocktail composition able to efficiently release the dried activity from the anion-exchange resin. This step is of crucial importance, because it particularly affects the final RCY and thus the success of the entire method. In this context, the elution cocktail used by the Munich Method (83 μmol KOH and 91 μmol Kryptofix® 222 in 500 μL MeCN) represents an efficient composition, as it allows for an almost quantitative recovery (98.3+0.6%, n=9). Small volumes (500-1000 μL) of MeCN and DMSO were considered for the elution of dried [¹⁸F]fluoride. The subsequent choice of the eluting salt was restricted by the solubility in the mentioned dipolar aprotic media. With the aim to avoid the need of toxic Kryptofix® 222 for metal ion complexation, ammonium or tetraalkylammonium were selected as salt cations. As corresponding counterions, several species including triflate, iodide, acetate and formate were investigated with respect to their ability to displace [¹⁸F]fluoride from the QMA resin (Table 1). Particular attention was paid to the anion basicity, which had to be considerably lower when compared to hydroxide. Such condition was supposed to be the key to omit additional neutralization of the eluate before radiolabeling. Nevertheless, the eluate maintains a slightly basic character through trace amounts of carbonate, which are always co-eluted from the QMA cartridge together with the [¹⁸F]fluoride. Entries 1 to 5 in the following table are provided as reference examples for comparative purposes.

TABLE 1
Recovery of dried [¹⁸F]fluoride from the Sep-Pak ® QMA
Carbonate cartridge (46 mg) using various
elution cocktails composed of ammonium or tetraalkylammonium
salts dissolved in dipolar aprotic media.
Before elution, activity was dried with air, MeCN (10 mL) and air again.

| | | | | | | |
|---|---|---|---|---|---|---|
| entry | salt | n(salt) [μmol] | solvent | V(solvent) [μL] | recovery [%] | n |

| | | | | | | |
|---|---|---|---|---|---|---|
| 1 | NBu₄OTf | 634 | MeCN | 500 | 0.4 | 1 |
| 2 | NBu₄l | 635 | MeCN | 500 | 2.3 | 1 |
| 3 | NH₄l | 638 | DMSO | 500 | 46.7 | 1 |
| 4 | NMe₄OAc | 633 | DMSO | 500 | 9.2 | 1 |
| 5 | NH₄OAc | 639 | DMSO | 500 | 77.4 | 1 |
| 6 | NH₄HCOO | 645 ± 1 | DMSO | 500 | 87.4 ± 4.0 | 2 |
| 7 | NH₄HCOO | 634 ± 3 | DMSO | 1000 | 89.7 ± 2.1 | 3 |

A high molar concentration of salt, which was approximately equal in all entries, was used in order to facilitate the release of dried [¹⁸F]fluoride. However, the investigated solutions with tetrabutylammonium salts in MeCN (entries 1 and 2) revealed to be both unsuitable as eluents. For the latter salt, the use of its ammonium analog in combination with DMSO as solvent (entry 3) significantly increased the elution efficiency to about 47%. A similar effect was observed for the evaluated acetate salts. While a solution of NMe₄OAc in DMSO (entry 4) only marginally displaced the activity from the QMA resin, the elution cocktail composed of NH₄OAc in DMSO (entry 5) achieved a recovery of over 77%. Replacing the acetate with formate proved to be even more advantageous. Hence, elution efficiency increased to almost 90% when using NH₄HCOO in DMSO (entries 6 and 7). Doubling the DMSO volume (entry 7) resulted in a slightly improved [¹⁸F]fluoride recovery. As the increase through higher eluent volume was not worth to slow down the concentration-dependent isotopic exchange rate in a subsequent radiofluorination with Silicon-based Fluoride Acceptors, the solvent amount was kept to 500 μL.

The correlation between molar amount of applied NH₄HCOO in DMSO and its respective [¹⁸F]fluoride elution capacity was elucidated in a further optimization study (Table 2). In this experiment series, drying of the QMA-bound activity was performed with DMSO (8 mL) in order to avoid the need of different solvents. Since dissolving 634 μmol of NH₄HCOO in 500 μL DMSO afforded an almost saturated solution, only lower or equal molar amounts were investigated. Entry 1 is provided as a reference example for comparative purposes.

TABLE 2
Recovery of dried [¹⁸F]fluoride from the Sep-Pak ® QMA
Carbonate cartridge (46 mg) using elution cocktails
with various amounts of NH₄HCOO dissolved in DMSO (500 μL).
Before elution, activity was dried with air, DMSO (8 mL) and air again.

| | | n(salt) | | V(solvent) | recovery | |
|---|---|---|---|---|---|---|
| entry | salt | [μmol] | solvent | [μL] | [%] | n |
| 1 | NH₄HCOO | 0 | DMSO | 500 | 0.1 ± 0.0 | 3 |
| 2 | NH₄HCOO | 79 | DMSO | 500 | 65.2 ± 1.1 | 3 |
| 3 | NH₄HCOO | 159 | DMSO | 500 | 74.2 ± 0.7 | 3 |
| 4 | NH₄HCOO | 396 | DMSO | 500 | 83.6 ± 0.6 | 3 |
| 5 | NH₄HCOO | 634 | DMSO | 500 | 88.4 ± 2.2 | 75 |

The elution efficiency increased with the rise in salt amount and reached a maximum of more than 88% when 634 μmol of NH₄HCOO were applied (entry 5). The [¹⁸F]fluoride recovery was consistent with the previously determined value using a comparable elution cocktail composition (Table 1, entry 6). Consequently, the choice of dipolar aprotic solvent (10 mL MeCN or 8 mL DMSO) for prior [¹⁸F]fluoride drying on the QMA cartridge seemed not to affect the elution step.

With the aim of further increasing the [¹⁸F]fluoride recovery, the impact of defined water amounts in the elution cocktail was studied (Table 3). The beneficial effect on the elution efficiency of aprotic eluents with additional water content was previously demonstrated by different groups. Brichard et Aigbirhio, for example, applied elution cocktails with 78 μmol of NEt₄HCO₃ dissolved in 1 mL of an aprotic solvent (MeCN, DMSO or DMF) with up to 5% water.¹⁴ Both authors observed a gradual increase in [¹⁸F]fluoride recovery correlating with the concentration of water.¹⁴ The same effect was also reported by Inkster et al. who illustrated that consistently higher elution efficiencies were observable when using various tetraethylammonium salts in solutions of MeCN or DMSO with increasing water content.¹⁵ However, it had to be kept in mind that the increased [¹⁸F]fluoride recovery through water addition came at the expense of the eluate reactivity. To estimate the advantage of a defined water content in the elution cocktail, it was crucial to determine the achievable RCY using the obtained eluate. The clinically established PSMA ligand ^{nat}Ga-rhPSMA-7.3 bearing a Silicon-based Fluoride Acceptor was therefor used as a model compound.

TABLE 3
Recovery of dried [¹⁸F]fluoride from the Sep-Pak ® QMA
Carbonate cartridge (46 mg) using elution cocktails composed of
NH₄HCOO (634 μmol) dissolved in DMSO with various water contents
(500 μL) and RCYs for the subsequent radiofluorination of
^{nat}Ga-rhPSMA-7.3 with corresponding eluates. Before elution,
activity was dried with air, DMSO (8 mL) and air again. Radiolabeling
was performed by combining the recovered eluate with
^{nat}Ga-rhPSMA-7.3 (1 m_{M} in DMSO, 150 μL, 150 nmol)
and incubating the mixture for 5 min at rt. The reaction mixture was
afterwards diluted with PBS (pH = 3, 10 mL) and loaded onto an Oasis ®
HLB cartridge (30 mg). After rinsing the cartridge with PBS (10 mL),
the purified tracer was eluted with EtOH/water (1:1, v/v, 300 μL).

| entry | water content (v/v) [%] | recovery [%] | n | RCY [%] | n |
|---|---|---|---|---|---|
| 1 | 0 | 88.4 ± 2.2 | 75 | 78.4 ± 0.4 | 2 |
| 2 | 1 | 92.7 ± 0.6 | 3 | 77.3 ± 1.5 | 3 |
| 3 | 2 | 94.2 ± 0.9 | 3 | 80.3 ± 1.2 | 3 |
| 4 | 4 | 94.8 ± 1.7 | 5 | 73.4 ± 3.9 | 4 |
| 5 | 5 | 95.3 ± 0.8 | 5 | 78.1 ± 6.0 | 5 |
| 6 | 10 | 95.6 ± 0.9 | 4 | 69.9 ± 7.7 | 4 |

In line with the aforementioned observations, the elution efficiency was found to further grow by the addition of water to the elution cocktail. While an eluent with 1% of water content demonstrated [¹⁸F]fluoride recovery of almost 93% (entry 2), the anhydrous analog (entry 1) released about 88% of trapped activity. The most efficient [¹⁸F]fluoride disposal (more than 95%) was determined using the elution cocktail with 10% of its volume corresponding to water (entry 6). With the aim to assess the eluate reactivity, subsequent ¹⁸F-labeling of ^{nat}Ga-rhPSMA-7.3 was conducted at rt for 5 min. Interestingly, RCYs for the radiofluorination reaction were in a comparable range when using eluates with water content up to 2% (entries 1, 2 and 3). Higher [¹⁸F]fluoride recoveries were consequently relativized by lower eluate reactivity due to the amount of water. Radiofluorination reactions involving eluates with even higher water contents tended to give lower RCYs (entries 4 and 6) and were generally less reproducible (entries 5 and 6). Since the addition of water to the elution cocktail is of no significant advantage, the eluent is preferably kept in its anhydrous composition (entry 1).

A scheme for the preferred [¹⁸F]fluoride preparation approach applied in the context of the present invention and subsequent application of the [¹⁸F]fluoride eluate for radiofluorination of a Silicon-based Fluoride Acceptor is provided in FIG. 2. The Figure illustrates the preferred [¹⁸F]fluoride preparation according to the present invention (step 1-3), subsequent application for radiofluorination of a Silicon-based Fluoride Acceptor-bearing compound (step 4-5) and final radiotracer purification via solid-phase extraction (step 6-9).

### Radiofluorination Methods

Radiofluorination of ^{nat}Ga-rhPSMA-7.3 in direct comparison with the Munich Method

In order to assess the performance of the present invention, a direct comparison with the established Munich Method regarding activity recovery and RCY for the subsequent radiofluorination of ^{nat}Ga-rhPSMA-7.3 was carried out (Table 4). For this purpose, ^{nat}Ga-rhPSMA-7.3 was ¹⁸F-labeled (GR1) with partly neutralized Munich eluate (GP2 and GE2) and thereafter purified by solid-phase extraction (GW1). In addition, ^{nat}Ga-rhPSMA-7.3 was radiofluorinated (GR1) with [¹⁸F]fluoride prepared by the present invention (GP1 and GE1) and subsequently purified via solid phase extraction (GW1). Entry 1 in the table is provided as a reference example for comparative purposes.

TABLE 4
Recovery of dried [¹⁸F]fluoride from the Sep-Pak ® QMA Carbonate
cartridge (46 mg) using the Munich Method or the present invention and RCYs for the
subsequent radiofluorination of ^{nat}Ga-rhPSMA-7.3 with corresponding eluates.

| entry | elution cocktail composition | recovery [%] | n | RCY [%] | n |
|---|---|---|---|---|---|
| 1 | KOH (83 μmol) & Kryptofix ® 222 (91 μmol) | 98.3 ± 0.6 | 9 | 74.9 ± 0.5 | 2 |
| | in MeCN (500 μL) | | | | |
| 2 | NH₄HCOO (634 μmol) in DMSO (500 μL) | 88.4 ± 2.2 | 75 | 78.4 ± 0.4 | 2 |

With respect to the elution efficiency, the Munich elution cocktail (entry 1) proved to exceed the eluent of the present invention (entry 2) by about 10%. Noteworthy, despite the considerably lower [¹⁸F]fluoride recovery for the present invention, RCYs of [¹⁸F]^{nat}Ga-rhPSMA-7.3 for both methods were found to be approximately the same. Thus, the labeling environment of the eluate afforded by the present invention presumably favors the isotopic exchange reaction in an extend even compensating the lower elution efficiency.

### Radiofluorination of a Base-Sensitive Compound Bearing a Silicon-Based Fluoride Acceptor

A base-sensitive compound bearing a Silicon-based Fluoride Acceptor was at first ¹⁸F-labeled (GR2) with partly neutralized Munich eluate (GP2 and GE2) and thereafter purified by solid-phase extraction (GW1). Subsequent analysis via radio-RP-HPLC (FIG. 3, A), however, revealed not only the synthesis of desired product (t_{R}=9.6 min) but also the presence of a radiofluorinated impurity (t_{R}=10.1 min) in the final formulation. In contrast, radiofluorination of the same compound (GR2 and GW1) with [¹⁸F]fluoride prepared by the present invention (GP1 and GE1), resulted only in the formation of pure ¹⁸F-labeled product (FIG. 3, B). This experiment demonstrates that the labeling environment of the Munich eluate might be incompatible with base-sensitive structures and emphasizes the utility of the present invention. In particular, FIG. 3 shows Radio-RP-HPLC chromatograms (column I, 10→70% B in A, 15 min, 95% B in A, 5 min, t_{R}=9.6 min) of an ¹⁸F-labeled base-sensitive Silicon-based Fluoride Acceptor-bearing compound purified by solid-phase extraction. A) [¹⁸F]Fluoride preparation according to the Munich Method and subsequent partial neutralization. B) [¹⁸F]Fluoride preparation according to the present invention.

### Radiofluorination of a Silicon-Based Fluoride Acceptor-Bearing Folate Receptor-Alpha Ligand Under Heating

A Silicon-based Fluoride Acceptor-bearing Folate Receptor-alpha ligand was radiofluorinated with partly neutralized Munich eluate (GP2 and GE2) at rt (GR3) as well as 95° C. (GR4) and the respective product subsequently purified via solid-phase extraction (GW1). Radiofluorination at the same temperatures (rt, GR3 and 95° C., GR4) was repeated using [¹⁸F]fluoride prepared by the present invention (GP1 and GE1) followed by analogous product purification (GW1). Determined RCYs for radiofluorination of the Folate Receptor-alpha ligand under the conditions described above are summarized as follows (Table 5).

TABLE 5
RCYs for the radiofluorination of a Silicon-based Fluoride
Acceptor-bearing Folate Receptor-alpha ligand with
[¹⁸F]fluoride prepared by either the Munich Method or
by the present invention. ^{a} Radiofluorination for 10 min at rt.
^{b} Radiofluorination for 10 min at 95° C.

| | |
|---|---|
| Munich Method | present invention |

| | RCY ª | | RCY ^{b} | | RCY ª | | RCY ^{b} | |
|---|---|---|---|---|---|---|---|---|
| | [%] | n | [%] | n | [%] | n | [%] | n |
| Folate Receptor- | 36.0 ± 2.0 | 2 | 20.0 ± 1.7 | 2 | 22.3 ± 3.0 | 3 | 54.1 ± 9.6 | 4 |
| alpha ligand | | | | | | | | |

Radiofluorination at rt using [¹⁸F]fluoride prepared by the present invention resulted in a RCY of about 20%. The analogous reaction involving partly neutralized Munich eluate afforded the ¹⁸F-labeled ligand in higher RCY (36.0±2.0%). However, the situation reversed when the radiolabeling reaction was carried out at 95° C. In this case, ¹⁸F-labeling with partially neutralized Munich eluate gave a diminished RCY, while radiofluorination under heating of the eluate generated by the present invention proved to be highly efficient (54.1±9.6%). A comparative radio-RP-HPLC analysis of the final product formulations was conducted in order to elucidate these results (FIG. 4, A-D). In particular, FIG. 4 shows Radio-RP-HPLC chromatograms (column II, 10→70% B in A, 15 min, 95% B in A, 5 min, t_{R}=13.0 min) of the ¹⁸F-labeled Silicon-based Fluoride Acceptor-bearing Folate Receptor-alpha ligand purified by solid-phase extraction. A) [¹⁸F]Fluoride preparation according to the present invention and radiofluorination at rt. B) [¹⁸F]Fluoride preparation following the present invention and radiofluorination at 95° C. C) [¹⁸F]Fluoride preparation according to the Munich Method, subsequent partial neutralization of the eluate and radiofluorination at rt. D) [¹⁸F]Fluoride preparation following the Munich Method, subsequent partial neutralization of the eluate and radiofluorination at 95° C.

When a Folate Receptor-alpha ligand was radiofluorinated at rt (FIG. 4, A) or 95° C. (FIG. 4, B) with the eluate generated by the present invention, a pure ¹⁸F-labeled product was obtained after solid phase extraction. Repeating the experiment with partly neutralized Munich eluate provided the same outcome when radiofluorination occurred at rt (FIG. 4, C). In contrast, the purified radioligand formulation afforded by partly neutralized Munich eluate heated to 95° C. (FIG. 4, D) showed the additional formation of an unknown side-product (t_{R}=12.4 min). This finding indicates that the Munich eluate is incompatible with higher temperatures, presumably due to the associated increase in reactivity of its basic environment. Consequently, heating the reaction mixture as a means to enhance RCY is only applicable to the eluate prepared by the present invention. This circumstance allows to obtain generally higher RCYs for the radiofluorination of Silicon-based Fluoride Acceptors that are insensitive to heat.

Radiofluorination of various siPSMA ligands with smallest amounts of precursor

215 MBq of aq. [¹⁸F]fluoride was trapped onto the QMA, dried and eluted according to GP1 and GE1. The eluate was used for the radiofluorination of siPSMA-01 following GR5 and GW1. ¹⁸F-siPSMA-01 was produced with a RCY of 11.1% and a Aₘ of 47.8 GBq/μmol.

145 MBq of aq. [¹⁸F]fluoride was trapped onto the QMA, dried and eluted according to GP1 and GE1. The eluate was used for the radiofluorination of siPSMA-02 following GR6 and GW1.

¹⁸F-siPSMA-02 was produced with a RCY of 9.6% and a Aₘ of 27.9 GBq/μmol.

142 MBq of aq. [¹⁸F]fluoride was trapped onto the QMA, dried and eluted according to GP1 and GE1. The eluate was used for the radiofluorination of siPSMA-03 following GR6 and GW1.

¹⁸F-siPSMA-03 was produced with a RCY of 8.5% and a Aₘ of 24.2 GBq/μmol.

125 MBq of aq. [¹⁸F]fluoride was trapped onto the QMA, dried and eluted according to GP1 and GE1. The eluate was used for the radiofluorination of siPSMA-04 following GR6 and GW1.

¹⁸F-siPSMA-04 was produced with a RCY of 10.9% and a Aₘ of 27.1 GBq/μmol.

169 MBq of aq. [¹⁸F]fluoride was trapped onto the QMA, dried and eluted according to GP1 and GE1. The eluate was used for the radiofluorination of siPSMA-05 following GR6 and GW1.

¹⁸F-siPSMA-05 was produced with a RCY of 8.5% and a Aₘ of 28.7 GBq/μmol.

148 MBq of aq. [¹⁸F]fluoride was trapped onto the QMA, dried and eluted according to GP1 and GE1. The eluate was used for the radiofluorination of siPSMA-06 following GR6 and GW1.

¹⁸F-siPSMA-06 was produced with a RCY of 12.1% and a Aₘ of 35.9 GBq/μmol.

180 MBq of aq. [¹⁸F]fluoride was trapped onto the QMA, dried and eluted according to GP1 and GE1. The eluate was used for the radiofluorination of siPSMA-07 following GR6 and GW1.

¹⁸F-siPSMA-07 was produced with a RCY of 10.5% and a Aₘ of 37.9 GBq/μmol.

161 MBq of aq. [¹⁸F]fluoride was trapped onto the QMA, dried and eluted according to GP1 and GE1. The eluate was used for the radiofluorination of siPSMA-08 following GR5 and GW1.

¹⁸F-siPSMA-08 was produced with a RCY of 12.1% and a Aₘ of 38.9 GBq/μmol.

168 MBq of aq. [¹⁸F]fluoride was trapped onto the QMA, dried and eluted according to GP1 and GE1. The eluate was used for the radiofluorination of siPSMA-09 following GR6 and GW1.

¹⁸F-siPSMA-09 was produced with a RCY of 7.9% and a Aₘ of 26.6 GBq/μmol.

279 MBq of aq. [¹⁸F]fluoride was trapped onto the QMA, dried and eluted according to GP1 and GE1. The eluate was used for the radiofluorination of siPSMA-11 following GR5 and GW1.

¹⁸F-siPSMA-11 was produced with a RCY of 11.2% and a Aₘ of 62.5 GBq/μmol.

161 MBq of aq. [¹⁸F]fluoride was trapped onto the QMA, dried and eluted according to GP1 and GE1. The eluate was used for the radiofluorination of siPSMA-12D following GR6 and GW1. ¹⁸F-siPSMA-12D was produced with a RCY of 8.6% and a Aₘ of 27.7 GBq/μmol.

205 MBq of aq. [¹⁸F]fluoride was trapped onto the QMA, dried and eluted according to GP1 and GE1. The eluate was used for the radiofluorination of siPSMA-12L following GR5 and GW1. ¹⁸F-siPSMA-12L was produced with a RCY of 11.0% and a Aₘ of 45.2 GBq/μmol.

200 MBq of aq. [¹⁸F]fluoride was trapped onto the QMA, dried and eluted according to GP1 and GE1. The eluate was used for the radiofluorination of siPSMA-13 following GR6 and GW1.

¹⁸F-siPSMA-13 was produced with a RCY of 8.5% and a Aₘ of 33.8 GBq/μmol.

230 MBq of aq. [¹⁸F]fluoride was trapped onto the QMA, dried and eluted according to GP1 and GE1. The eluate was used for the radiofluorination of siPSMA-14 following GR5 and GW1.

¹⁸F-siPSMA-14 was produced with a RCY of 5.5% and a Aₘ of 25.2 GBq/μmol.

160 MBq of aq. [¹⁸F]fluoride was trapped onto the QMA, dried and eluted according to GP1 and GE1. The eluate was used for the radiofluorination of siPSMA-15 following GR6 and GW1.

¹⁸F-siPSMA-15 was produced with a RCY of 6.6% and a Aₘ of 21.2 GBq/μmol.

171 MBq of aq. [¹⁸F]fluoride was trapped onto the QMA, dried and eluted according to GP1 and GE1. The eluate was used for the radiofluorination of siPSMA-16 following GR6 and GW1.

¹⁸F-siPSMA-16 was produced with a RCY of 5.3% and a Aₘ of 18.2 GBq/μmol.

273 MBq of aq. [¹⁸F]fluoride was trapped onto the QMA, dried and eluted according to GP1 and GE1. The eluate was used for the radiofluorination of siPSMA-17 following GR5 and GW1.

¹⁸F-siPSMA-17 was produced with a RCY of 12.4% and a Aₘ of 58.8 GBq/μmol.

206 MBq of aq. [¹⁸F]fluoride was trapped onto the QMA, dried and eluted according to GP1 and GE1. The eluate was used for the radiofluorination of siPSMA-18D following GR5 and GW1. ¹⁸F-siPSMA-18D was produced with a RCY of 13.9% and a Aₘ of 57.3 GBq/μmol.

193 MBq of aq. [¹⁸F]fluoride was trapped onto the QMA, dried and eluted according to GP1 and GE1. The eluate was used for the radiofluorination of siPSMA-18L following GR5 and GW1. ¹⁸F-siPSMA-18L was produced with a RCY of 12.9% and a Aₘ of 49.8 GBq/μmol.

250 MBq of aq. [¹⁸F]fluoride was trapped onto the QMA, dried and eluted according to GP1 and GE1. The eluate was used for the radiofluorination of siPSMA-19 following GR6 and GW1.

¹⁸F-siPSMA-19 was produced with a RCY of 7.9% and a Aₘ of 39.7 GBq/μmol.

257 MBq of aq. [¹⁸F]fluoride was trapped onto the QMA, dried and eluted according to GP1 and GE1. The eluate was used for the radiofluorination of siPSMA-20 following GR5 and GW1.

¹⁸F-siPSMA-20 was produced with a RCY of 8.3% and a Aₘ of 42.5 GBq/μmol.

223 MBq of aq. [¹⁸F]fluoride was trapped onto the QMA, dried and eluted according to GP1 and GE1. The eluate was used for the radiofluorination of siPSMA-21 following GR5 and GW1.

¹⁸F-siPSMA-21 was produced with a RCY of 11.2% and a Aₘ of 50.1 GBq/μmol.

### TERMS AND ACRONYMS

abs. absolute
add. additional
Aₘ molar activity
anhyd. anhydrous
approx. approximately
aq. aqueous
Bu butyl
DMF N,N-dimethylformamide
DMSO dimethyl sulfoxide
Et ethyl
EtOH ethanol
Kryptofix® 222 4,7,13,16,21,24-Hexaoxa-1,10-diazabicyclo [8.8.8]hexacosane
HPLC high performance liquid chromatography
MeCN acetonitrile
Me methyl
OAc acetate
OTf triflate
PET positron emission tomography
PBS phosphate-buffered saline
precon. preconditioned
PSMA prostate-specific membrane antigen
RCC radiochemical conversion
RCY radiochemical yield
RP reversed-phase
rt room temperature
t_{R} retention time
TFA trifluoroacetic acid
UV ultraviolet
Vis visible

### BRIEF DESCRIPTION OF THE FIGURES

FIG. 1: General scheme for the [¹⁸F]fluoride preparation according to the Munich Method (step 1-3), subsequent application for radiofluorination of a Silicon-based Fluoride Acceptor-bearing compound (step 4-5) and final radiotracer purification via solid-phase extraction (step 6-9).

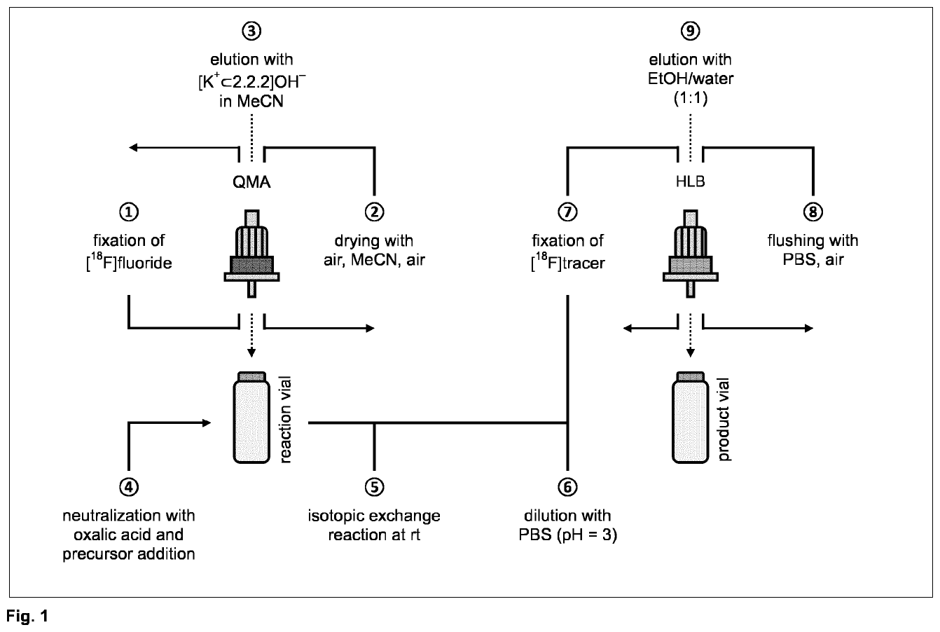

FIG. 2: Scheme for the preferred [¹⁸F]fluoride preparation according to the present invention (step 1-3), subsequent application for radiofluorination of a Silicon-based Fluoride Acceptor-bearing compound (step 4-5) and final radiotracer purification via solid-phase extraction (step 6-9).

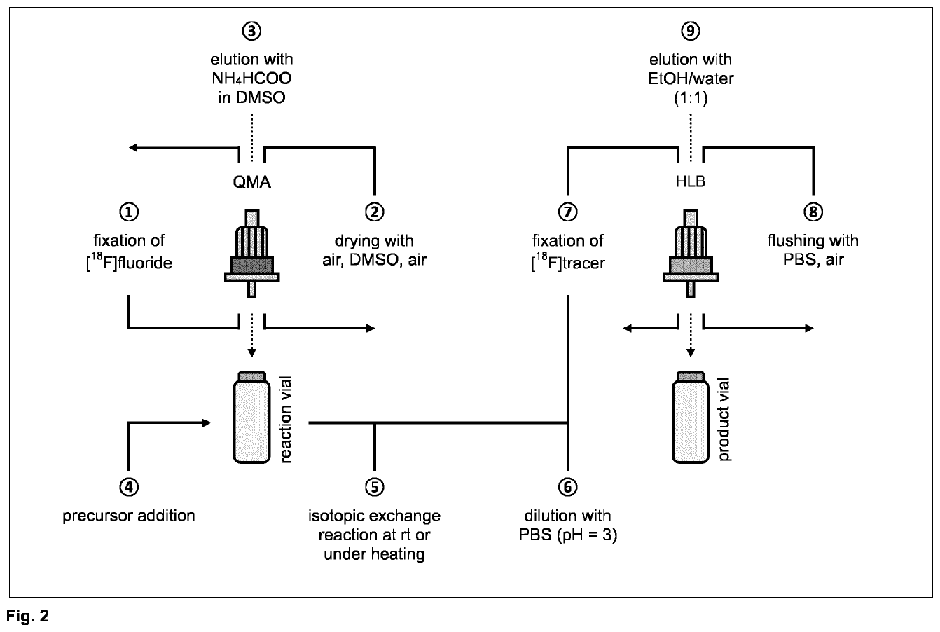

FIG. 3: Radio-RP-HPLC chromatograms (column I, 10→70% B in A, 15 min, 95% B in A, 5 min, t_{R}=9.6 min) of an 18F-labeled base-sensitive Silicon-based Fluoride Acceptor-bearing compound purified by solid-phase extraction. A) [¹⁸F]Fluoride preparation according to the Munich Method and subsequent partial neutralization. B) [¹⁸F]Fluoride preparation according to the present invention.

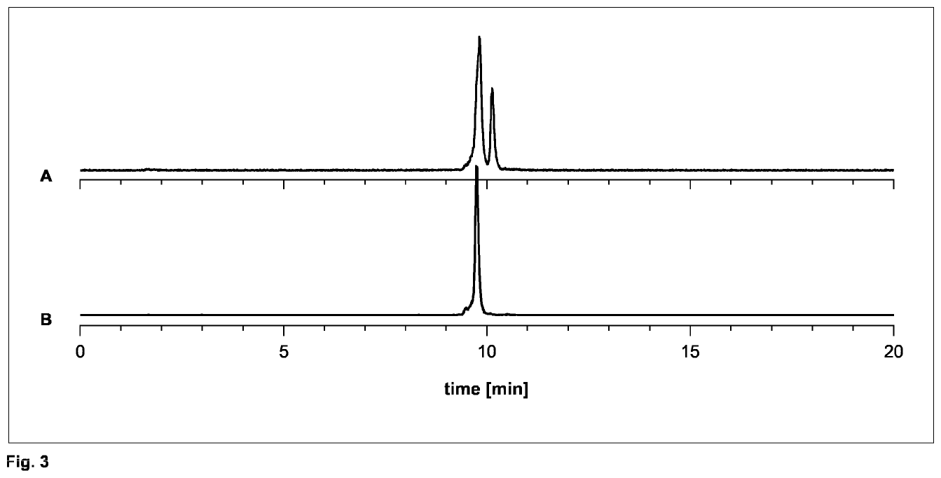

FIG. 4: Radio-RP-HPLC chromatograms (column II, 10→70% B in A, 15 min, 95% B in A, 5 min, t_{R}=13.0 min) of the 18F-labeled Silicon-based Fluoride Acceptor-bearing Folate Receptor-alpha ligand purified by solid-phase extraction. A) [¹⁸F]Fluoride preparation according to the present invention and radiofluorination at rt. B) [¹⁸F]Fluoride preparation following the present invention and radiofluorination at 95° C. C) [¹⁸F]Fluoride preparation according to the Munich Method, subsequent partial neutralization of the eluate and radiofluorination at rt. D) [¹⁸F]Fluoride preparation following the Munich Method, subsequent partial neutralization of the eluate and radiofluorination at 95° C.

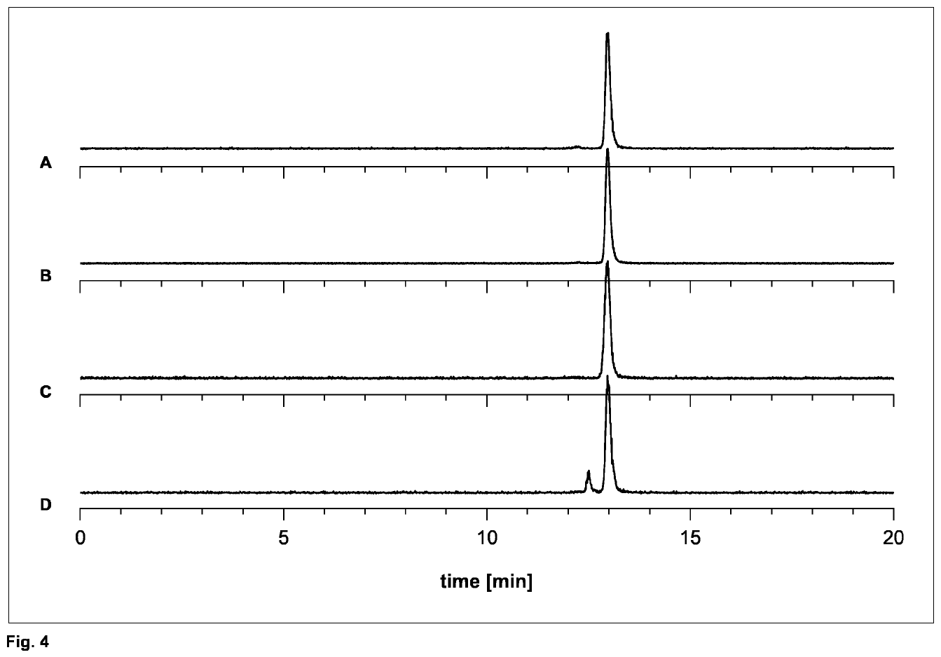

1. A method for the preparation of a composition comprising dissolved [¹⁸F]fluoride ions, said method comprising the steps of
providing an aqueous solution comprising water and [¹⁸F]fluoride ions;
passing the aqueous solution through a solid phase extraction device comprising an anion exchange resin in order to trap [¹⁸F]fluoride ions on the anion exchange resin and to separate the [¹⁸F]fluoride ions trapped on the anion exchange resin from water;
eluting [¹⁸F]fluoride ions from the anion exchange resin by passing an elution composition comprising an organic solvent and a salt of an alkanoic acid through the solid phase extraction device;
obtaining a composition as an eluate which comprises the organic solvent, the salt of the alkanoic acid, and dissolved [¹⁸F]fluoride ions.

2. The method according to claim 1, which further comprises a step of purging the solid phase extraction device comprising the trapped [¹⁸F]fluoride ions on the anion exchange resin with a gas after the aqueous solution has been passed through the device.

3. The method according to claim 1 or 2, which further comprises a step of rinsing the anion exchange resin comprising the trapped [¹⁸F]fluoride ions with an organic solvent prior to eluting [¹⁸F]fluoride ions from the anion exchange resin.

4. The method according to any one of claims 1 to 3, wherein the elution composition comprises a salt of an alkanoic acid represented by formula (A-1): 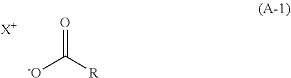
wherein:
X⁺ is selected from an ammonium cation, an alkyl ammonium cation and a cryptate of an alkali or alkaline earth metal cation;
R is H, a linear or branched C1 to C20 alkyl group.

5. The method according to any one of claims 1 to 4, wherein the salt of the alkanoic acid comprises a formate salt.

6. The method according to any one of claims 1 to 5, wherein the concentration of the salt of the alkanoic acid in the elution composition is in the range of 0.1 to 1.5 mol/l.

7. The method according to any one of claims 1 to 6, wherein the organic solvent comprised by the elution composition comprises a polar aprotic organic solvent, preferably a solvent selected from dimethyl sulfoxide and acetonitrile.

8. The method according to any one of claims 1 to 7, wherein each organic solvent used in the method is an anhydrous organic solvent.

9. The method according to any one of claims 1 to 8, wherein the elution composition further comprises an organic compound to be radiofluorinated.

10. A method for the preparation of a radiofluorinated organic compound, wherein the method comprises the steps of
preparing a composition by the method in accordance with any of claims 1 to 8, which composition comprises an organic solvent, a salt of an alkanoic acid and dissolved [¹⁸F]fluoride ions; and
contacting the organic compound to be radiofluorinated with the composition to allow the organic compound to undergo a radiofluorination reaction with a [¹⁸F]fluoride ion comprised in the composition.

11. The method according to claim 10, wherein the composition that is obtained as an eluate in accordance with the method as defined in any of claims 1 to 8 is directly contacted with the organic compound to be radiofluorinated without any modification of the composition.

12. A method for the preparation of a radiofluorinated organic compound, wherein the method comprises the steps of
preparing a composition by the method in accordance with claim 9, which composition comprises an organic solvent, a salt of an alkanoic acid, dissolved [¹⁸F]fluoride ions and an organic compound to be radiofluorinated; and
allowing the organic compound to undergo a radiofluorination reaction with a [¹⁸F]fluoride ion comprised in the composition.

13. The method according to any one of claims 10 to 12, wherein the organic compound to be radiofluorinated comprises a non-radiofluorinated silicon-based fluoride acceptor (SiFA) moiety with a functional group represented by formula (S-1): 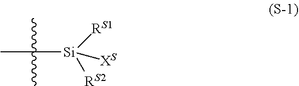
wherein:
X^{S} is ¹⁹F, OH or H,
R^{S1} and R^{S2} are independently a linear or branched C3 to C10 alkyl group, and wherein the waved line marks the bond which attaches the functional group to the remainder of the organic compound;
and wherein the radiofluorination reaction involves an exchange of the group X^{S} by ¹⁸F.

14. A composition comprising an organic solvent, a salt of an alkanoic acid and dissolved [¹⁸F]fluoride ions.

15. The composition in accordance with claim 14, wherein the organic solvent comprises a polar aprotic organic solvent selected from dimethyl sulfoxide and acetonitrile, and the salt of an alkanoic acid comprises ammonium formate.
